(19) United States
(12) Patent Application Publication
(10) Pub. No.: US 2026/0290626 A1
(43) Pub. Date: Sep. 24, 2026

(54) SCIENTIFIC PROOF OF GOD (SPOG): SYSTEMS AND METHODS FOR VERIFYING INTENTIONAL BIOLOGICAL DESIGN IN HUMAN DEVELOPMENT

(71) Applicant: Don Karl Juravin, San Antonio, FL (US)

(72) Inventor: Don Karl Juravin, San Antonio, FL (US)

(21) Appl. No.: 19/462,549

(22) Filed: Jan. 28, 2026

Related U.S. Application Data
(63) Continuation-in-part of application No. 18/092,272, filed on Dec. 31, 2022, now abandoned.
(63) Continuation-in-part of application No. 19/308,719, filed on Aug. 25, 2025.
(63) Continuation-in-part of application No. 19/446,170, filed on Jan. 12, 2026.

(51) Int. Cl. G16Z 99/00; G06F 40/242; G16H 50/70

(52) U.S. Cl. CPC G16Z 99/00; G06F 40/242; G16H 50/70

(57) ABSTRACT

A system and method verify intentional biological design in human development by correlating fixed developmental timelines with encoded linguistic-numeric values derived from original Hebrew biological terminology using an established decoding scheme. The disclosed framework, referred to as the Scientific Proof of God (SPOG), provides a structured approach for computing numeric values of Hebrew organ and process terms and mapping those values to empirically observable developmental milestones at which an organ becomes functionally operative. The system further provides calibration utilities for clinical research by replacing probabilistic conception-timing estimates with fixed reference benchmarks; provides an objective method for determining fetal developmental status based on functional readiness; and provides visualization interfaces that present cross-domain consistency between linguistic encodings and biological outcomes. The system thereby supplies a reproducible verification framework distinguishing intentional biological design from probabilistic or Darwinian evolutionary models.

### PRIOR HISTORY

This patent application is a Continuation-in-Part patent application claiming the benefit of pending U.S. patent application Ser. No. 18/092,272 filed in the United States Patent and Trademark Office (USPTO) on 31 Dec. 2022; and a Continuation-in-Part patent application claiming the benefit of pending U.S. patent application Ser. No. 19/308,719 filed in the USPTO on 25 Aug. 2025; and a Continuation-in-Part patent application claiming the benefit of pending U.S. patent application Ser. No. 19/446,170 filed in the USPTO on 12 Jan. 2026, the specifications and drawings of which applications are hereby incorporated by reference.

### FIELD OF THE SUBJECT MATTER

The present disclosure relates to developmental biology, embryology, medical calibration, and scientific verification systems. More particularly, the disclosure relates to systems and methods for computing linguistic-numeric values from original Hebrew biological terminology using an established decoding scheme and correlating those computed values to empirically observable human developmental milestones, including gestational timing and organ functionality benchmarks, to verify intentional biological design and to provide practical clinical and research utilities.

### BRIEF DESCRIPTION OF THE PRIOR ART

Human embryological development contains numerous biological constants that remain stable across populations, environments, and historical periods, including gestational duration and organ-maturity timelines. Modern embryology measures these parameters using observational and statistical tools that often rely on estimated conception dates and exhibit variability across datasets. Separately, the Hebrew language possesses a historically stable alphabetic structure consisting of twenty-two letters. U.S. Pat. No. 12,400,087 established a reproducible method (the code2GOD Scientific System Decoder) for assigning fixed numeric values to these letters to derive secondary or encoded meaning from Hebrew lexical items.

There remains a need for analytic tools that integrate these two domains—linguistic encoding and biological measurement—in order to (i) identify possible structured relationships, (ii) quantify the degree of correlation, (iii) present these relationships in comprehensible visual form, and (iv) calibrate biomedical measurements using fixed numeric constants derived from linguistic encodings. The presently disclosed subject matter fulfills these needs by providing a comprehensive analytical and visualization framework that maps Hebrew linguistic values to biological, developmental, and conceptual data; verifies the stability of developmental constants; and displays all encoded correlations in a unified scientific interface.

Accurate determination of conception timing and organ developmental milestones is foundational to prenatal medicine, embryological research, and clinical decision-making. However, modern clinical practice commonly relies on indirect and probabilistic estimation methods, including patient-reported last menstrual period, assumed cycle length, and statistical averaging. These methods can introduce uncertainty of multiple days and, in common research practice, may yield developmental assessments expressed in broad ranges spanning one to three weeks. Such variability reduces the precision and reproducibility of clinical studies and complicates calibration of observed developmental phenomena against any fixed reference standard.

In parallel, Darwinian evolutionary theory asserts that biological structures and developmental characteristics are subject to gradual change over extended timescales due to random mutation and selection pressures. Under such models, fixed and invariant biological timelines governing complex multistage processes—such as human gestation and organ maturation—would be expected to exhibit measurable drift when considered across long historical time spans and diverse populations. Yet core aspects of human development are observed to be remarkably stable.

The present disclosure addresses these deficiencies by introducing a computational and evidentiary framework in which fixed developmental benchmarks are identified and validated across multiple organs and developmental stages using encoded linguistic-numeric values derived from original Hebrew biological terminology. These encoded values are computed using an established decoding scheme and correlated to empirically observable milestones of organ functionality. The repeated and systematic alignment between computed linguistic-numeric values and fixed biological timelines across multiple organ systems substantially exceeds what would be expected from statistical coincidence, thereby supporting verification of intentional biological design and enabling practical utilities in medical calibration, fetal developmental assessment, and visualization.

### GENERAL DESCRIPTIONS AND SUMMARY

The presently disclosed subject matter provides a set of analytical methods, systems, and visualization interfaces for identifying, measuring, and presenting structured numeric relationships embedded within Hebrew lexical data and their correspondence to empirically verified biological, developmental, and scientific constants. The presently disclosed subject matter builds upon the numeric assignments defined in U.S. Pat. No. 12,400,087 (code2GOD Scientific System Decoder) by applying those assignments to Hebrew terms related to organs, developmental processes, generative concepts, anatomical symmetry, gestation, and textual constructs.

Through these methods, the presently disclosed subject matter produces exact equivalences between computed code2GOD values and the number of days from conception to functional maturity for numerous human organs, including heart, kidney, brain, stomach, bone, and sensory structures. The presently disclosed subject matter further incorporates these correlations into visual embodiments—such as large-format scientific artworks—that integrate encoded linguistic values, biological-development timelines, conceptual encodings, and complete textual rendering of the Hebrew Biblical corpus.

The presently disclosed subject matter provides (i) methods for extracting encoded scientific information from Hebrew language structures, (ii) systems for comparing encoded constants with medical and embryological measurements, (iii) procedures for verifying textual integrity across Hebrew manuscripts, and (iv) visualization interfaces that present the encoded linguistic-biological relationships for scientific, medical, and educational use.

There is provided according to a first aspect of the presently disclosed subject matter a scientific verification system for identifying intentional biological design in human development by correlating fixed biological timelines with encoded linguistic and numeric representations. The scientific verification system comprises a Hebrew lexical repository storing a plurality of original, unvocalized Hebrew terms corresponding to human organs and human biological processes; and a decoding module configured to apply a letter-to-number assignment defined by a patented code2GOD Scientific System Decoder to each of the stored Hebrew terms, and to compute, for each stored Hebrew term, a respective aggregate numeric value that is a function of numeric values assigned to constituent Hebrew letters of the term.

The scientific verification system further comprises a developmental benchmark repository storing a plurality of fixed human developmental benchmarks expressed in exact temporal units and including at least (i) a gestational-duration benchmark and (ii) organ-functionality benchmarks each associated with a respective organ function; and a correlation engine configured to (i) select a subset of the stored Hebrew terms, (ii) retrieve the aggregate numeric values for the selected subset, (iii) retrieve corresponding benchmarks from the developmental benchmark repository, and (iv) determine, for each selected Hebrew term, whether the aggregate numeric value equals or satisfies a predefined correspondence rule with a selected developmental benchmark.

The scientific verification system further comprises a multi-organ aggregation module configured to aggregate correlation results across a plurality of distinct organs and biological processes to generate a combined correspondence set comprising multiple independent correspondences; and a statistical evaluation module configured to compute at least one statistical indicator quantifying likelihood that the combined correspondence set occurs by chance, wherein the statistical indicator is computed based on the number of correspondences, the size of the search space of evaluated Hebrew terms, and the number of benchmark categories evaluated.

The scientific verification system further comprises an invariance validation module configured to validate that at least a subset of the fixed human developmental benchmarks remain invariant across at least two independent population datasets or clinical references, thereby confirming resistance of the validated benchmarks to developmental drift; and a design-verification decision module configured to generate a design-verification output by applying predefined acceptance criteria that require (i) said combined correspondence set, (ii) said statistical indicator satisfying a non-coincidence threshold, and (iii) said invariance validation.

The scientific verification system further comprises an output interface configured to generate at least one machine-readable or human-readable report comprising: identified Hebrew terms, computed aggregate numeric values, matched developmental benchmarks, the statistical indicator, the invariance validation result, and the design-verification output, wherein the scientific verification system produces a reproducible verification result indicating intentional biological design when the predefined acceptance criteria are satisfied.

In some embodiments, the human gestation period is fixed and invariant across human history. In some embodiments, organ development timelines are encoded in Hebrew organ terminology. In some embodiments, heart functionality is achieved at a fixed developmental point corresponding to four-chamber operation. In some embodiments, kidney functionality is achieved at a fixed developmental point corresponding to filtration capability. In some embodiments, brain functionality corresponds to a fixed developmental milestone. In some embodiments, organ development timelines remain unchanged across extended historical timescales. In some embodiments, these timelines are shielded from evolutionary modification. In some embodiments, the fixed developmental constraints refute a purely Darwinian single-cell origin of human life. In some embodiments, a distinction is established between a passive creator model and an active designer model.

In some embodiments, the gestational-duration benchmark is validated against multiple independent medical references and historical population datasets to confirm invariance of human pregnancy duration. In some embodiments, the organ-functionality benchmarks comprise a plurality of benchmarks each corresponding to a distinct organ and a distinct biological function defining functional operability of said organ. In some embodiments, a Hebrew term corresponding to the heart yields an aggregate numeric value that matches a developmental benchmark at which four-chamber cardiac circulation becomes functionally operative. In some embodiments, a Hebrew term corresponding to the kidneys yields an aggregate numeric value that matches a developmental benchmark at which renal filtration of blood plasma is functionally established.

In some embodiments, a Hebrew term corresponding to the brain yields an aggregate numeric value that matches a developmental benchmark associated with a defined neurological maturation event. In some embodiments, the invariance validation module confirms that the fixed developmental benchmarks remain constant across at least two distinct human populations separated geographically or temporally. In some embodiments, the confirmed invariance is inconsistent with developmental drift predicted by evolutionary mutation-based models. In some embodiments, the statistical evaluation module computes a cumulative probability value based on multiple independent organ correspondences to determine non-randomness of the correlations. In some embodiments, the design-verification decision module distinguishes an actively maintained biological design model from a passive origin model based on sustained benchmark invariance.

There is provided according to a second aspect of the presently disclosed subject matter a medical calibration system for determining organ functionality stages in human development based on designer-defined timelines rather than probabilistic estimation. The medical calibration system comprising a benchmark dataset storing fixed organ-functionality timelines expressed in exact temporal units, each fixed organ-functionality timeline being derived from encoded linguistic-numeric correlations generated using a decoding scheme defined by a patented code2GOD Scientific System Decoder and associating a respective organ with a respective functional readiness milestone.

The medical calibration system further comprises a clinical intake module configured to receive clinical developmental observations for a subject, including at least one of ultrasound measurements, biomarker measurements, imaging findings, or physician-recorded developmental assessments, wherein the clinical developmental observations are associated with an estimated conception timing determined by at least one probabilistic estimation method comprising last-menstrual-period dating, assumed cycle-length dating, or population-average gestational dating.

The medical calibration system further comprises a benchmark alignment engine configured to align the received clinical developmental observations to the benchmark dataset by mapping observed indicators of organ development to the fixed organ-functionality timelines for corresponding organs; and an estimation-error quantification module configured to compute, for each mapped organ, a deviation value representing an offset between (i) an observation-based developmental timing inferred under the probabilistic estimation method and (ii) the fixed organ-functionality timeline associated with the organ in the benchmark dataset, wherein the deviation value is expressed in exact temporal units.

The medical calibration system further comprises a calibration output generator configured to generate one or more calibration outputs comprising: (i) a corrected developmental-stage classification for the subject anchored to the benchmark dataset, (ii) an error band indicating reliability of the probabilistic estimation method for the received observations, and (iii) an identification of organs for which the received observations materially conflict with the fixed organ-functionality timelines; and a research calibration module configured to apply the calibration output generator across a plurality of subjects to produce a study-level calibration report that quantifies temporal bias introduced by probabilistic conception estimation within clinical datasets; and an output interface configured to provide the corrected developmental-stage classification and the study-level calibration report for use in prenatal assessment and clinical research, wherein the medical calibration system replaces probabilistic estimation of conception timing with fixed organ-functionality benchmarks as a reference standard for determining organ functionality stages. In some embodiments, the medical calibration system is used to validate clinical research findings. In some embodiments, the calibration output generator produces a study-level correction factor quantifying temporal bias introduced by probabilistic conception estimation methods used in clinical datasets.

There is provided according to a third aspect of the presently disclosed subject matter a method for determining fetal life status based on the functional readiness of organs as defined by fixed developmental milestones. The method for determining fetal life status comprises the steps of: selecting a plurality of organs relevant to biological life readiness, the plurality including at least one organ selected from the group consisting of heart, brain, kidneys, lungs, and other organs essential to sustained biological function; and (b) retrieving, for each selected organ, a fixed functionality milestone from a benchmark dataset, each fixed functionality milestone being expressed in exact temporal units and derived from encoded linguistic-numeric correlations generated using a decoding scheme defined by a patented code2GOD Scientific System Decoder.

The method for determining fetal life status further comprises the steps of obtaining developmental observations for a subject indicative of the development state of each selected organ, wherein the developmental observations are independent of estimated conception timing and include at least one of imaging data, physiological measurements, or clinical indicators of organ function; and evaluating, for each selected organ, whether the developmental observations satisfy functional readiness criteria associated with the retrieved fixed functionality milestone for the organ.

The method for determining fetal life status further comprises the steps of aggregating organ-level evaluations to determine whether a predefined functional readiness condition is satisfied based on a combination of organs that collectively indicate biological life readiness; and accounting for postnatal maturation for organs whose functional completion occurs after birth by applying organ-specific postnatal benchmarks when evaluating functional readiness; and outputting a fetal life status determination that classifies developmental life status based on satisfaction of the predefined functional readiness condition rather than on chronological gestational age or probabilistic conception estimation, wherein the method provides an objective and reproducible determination of fetal life status anchored to fixed organ functionality milestones. In some applications, fetal life status is determined based on functional organ readiness rather than chronological age. In some applications, the step of determining fetal life status includes applying organ-specific postnatal functionality benchmarks for organs whose functional completion occurs after birth.

There is provided according to a fourth aspect of the presently disclosed subject matter a scientific visualization framework integrating biological, linguistic, and numeric data to visually represent intentional design in human development. The scientific visualization framework comprises a data integration module configured to ingest and normalize (i) fixed human developmental benchmarks expressed in exact temporal units, (ii) aggregate numeric values computed from Hebrew biological terms using a decoding scheme defined by a patented code2GOD Scientific System Decoder, and (iii) statistical validation outputs generated from multi-organ correspondence analysis; and a cross-domain mapping engine configured to associate encoded linguistic-numeric values with corresponding biological developmental milestones and to align said associations across multiple organs and developmental stages.

The scientific visualization framework further comprises a temporal rendering engine configured to generate visual timelines illustrating exact points of organ functionality onset and gestational progression based on the fixed human developmental benchmarks; a comparative model renderer configured to visually contrast probabilistic developmental estimation models with fixed benchmark-based developmental representations, thereby illustrating deviation between estimation-based and benchmark-based interpretations; and an invariance visualization module configured to visually indicate consistency of developmental benchmarks across populations, historical references, or datasets to demonstrate resistance to developmental drift.

The scientific visualization framework further comprises a user interaction interface configured to enable selection, exploration, and comparison of organs, timelines, and encoded values by a user; and an evidence presentation layer configured to generate visual artifacts suitable for scientific analysis, education, publication, or evidentiary presentation, wherein the scientific visualization framework provides an interpretable visual representation of invariant developmental consistency indicative of intentional biological design.

### BRIEF DESCRIPTION OF DRAWINGS

Other features and objectives of the systems/method/framework according to the presently disclosed subject matter will become more evident from a consideration of the following brief descriptions of patent drawings.

FIG. 1 depicts a prior-art style Hebrew letter-to-numeric value reference table, illustrating an exemplary letter-value assignment scheme used for converting Hebrew letters into numeric representations. This figure provides contextual support for the numeric encoding processes in support of the scientific verification system according to the presently disclosed subject matter.

FIG. 2 illustrates an exemplary computation in which numeric values associated with individual Hebrew letters are combined to yield an aggregate numeric value for a selected Hebrew term. This figure conceptually illustrates aggregation of encoded values in support of the scientific verification system according to the presently disclosed subject matter.

FIG. 3A illustrates an exemplary conceptual composition combining multiple encoded Hebrew terms with associated numeric values, shown as an illustrative aggregation of encoded linguistic components. This figure supports the multi-term aggregation and correlation concepts in support of the scientific verification system according to the presently disclosed subject matter.

FIG. 3B illustrates an exemplary conceptual relationship in which multiple encoded Hebrew terms are combined to produce an output concept, serving as an illustrative example of correlation output generation in support of the scientific verification system according to the presently disclosed subject matter.

FIG. 3C illustrates an exemplary encoded mapping of family-related biological terms and associated numeric values, provided as an additional example of encoded linguistic aggregation applied to biological vocabulary. This figure supports the use of biological lexical terms in support of the scientific verification system according to the presently disclosed subject matter.

FIG. 3D illustrates an exemplary encoded numeric relation involving a subtraction operation applied to encoded Hebrew terms, demonstrating computational processing of encoded linguistic values. This figure further supports computational correlation and output generation in support of the scientific verification system according to the presently disclosed subject matter.

FIG. 4 illustrates an exemplary conceptual progression linking encoded Hebrew terms associated with soil/land, human, and blood, showing relational mapping between biologically relevant lexical concepts. This figure supports the Hebrew lexical repository and relational analysis in support of the scientific verification system according to the presently disclosed subject matter.

FIG. 5 illustrates an exemplary encoded relation involving biological sustenance terminology and a computed numeric result, further demonstrating application of numeric operations to encoded Hebrew terms. This figure supports the numeric encoding and correlation processes in support of the scientific verification system according to the presently disclosed subject matter.

FIG. 6 illustrates an exemplary comparison between two encoded Hebrew terms associated with different conceptual meanings, showing numeric differentiation and comparison. This figure conceptually supports encoded term comparison and correlation in support of the scientific verification system according to the presently disclosed subject matter.

FIG. 7A illustrates a conceptual representation of completeness of the Hebrew alphabet, showing the full set of letters as a bounded system. This figure supports the definition of the Hebrew letter set used in numeric encoding and decoding in support of the scientific verification system and visually supported by the scientific visualization framework according to the presently disclosed subject matter.

FIG. 7B illustrates an exemplary equality between an encoded Hebrew term and a numeric phrase, serving as an example of encoded output reporting. This figure supports the scientific verification system and the scientific visualization framework according to the presently disclosed subject matter.

FIG. 8A illustrates an exemplary conceptual representation associating an encoded Hebrew term with a numeric biological constant, presented as a visualization of encoded biological correspondence. This figure supports the scientific visualization framework according to the presently disclosed subject matter.

FIG. 8B illustrates an exemplary simplified human body representation associated with a numeric biological constant, provided as a visualization example for presenting biological benchmarks. This figure supports the scientific visualization framework according to the presently disclosed subject matter.

FIG. 9A illustrates an exemplary representation of a Hebrew textual sequence associated with a numeric ratio approximation, presented as an illustrative example of numeric relationship analysis derived from Hebrew letter sequences. This figure supports the correlation concepts in support of the scientific verification system and the scientific visualization framework according to the presently disclosed subject matter.

FIG. 9B illustrates an exemplary mapping between an encoded numeric sentence value and a sequence of digits associated with a mathematical constant, presented as an example of correlation output visualization. This figure supports the correlation concepts in support of the scientific verification system and the scientific visualization framework according to the presently disclosed subject matter.

FIG. 10 illustrates an exemplary encoded presentation of a Hebrew term associated with ancestral or familial identifiers, provided as an illustrative encoded labeling example. This figure supports the scientific visualization framework and the encoded lexical context in support of the scientific verification system according to the presently disclosed subject matter.

FIG. 11A illustrates an exemplary geometric clustering visualization showing grouped elements arranged in a structured pattern. This figure supports visualization of grouping and aggregation patterns within the scientific visualization framework according to the presently disclosed subject matter.

FIG. 11B illustrates an alternate geometric clustering visualization showing a different grouping configuration. This figure further supports visualization of aggregation and pattern representation in support of the scientific visualization framework according to the presently disclosed subject matter.

FIG. 12 illustrates a stylized visual presentation of Hebrew letters arranged in a chart format, provided as a visual representation of the Hebrew letter set used for encoding and decoding. This figure supports the decoding context in support of the scientific verification system and the scientific visualization framework according to the presently disclosed subject matter.

FIG. 13 illustrates an exemplary fetal visualization annotated with organ-related encoded numeric values, conceptually mapping encoded Hebrew biological terms to organ-specific developmental timing benchmarks. This figure supports multi-organ correspondence in support of the scientific verification system; benchmark context in support of the medical calibration system; functional readiness considerations in support of the method for determining fetal life status; and visualization in support of the scientific visualization framework according to the presently disclosed subject matter.

FIG. 14 illustrates an exemplary pregnancy visualization including an encoded gestational term and an associated gestational-day value, provided as an illustrative representation of fixed gestational duration. This figure supports gestational benchmark correlation in support of the scientific verification system; fixed developmental benchmarks in support of the medical calibration system; and visualization in support of the scientific visualization framework according to the presently disclosed subject matter.

FIG. 15A illustrates an exemplary conception-stage visualization including encoded sperm and egg terminology with associated numeric values, conceptually depicting early developmental stages. This figure supports encoded biological term computation in support of the scientific verification system; benchmark context in support of the medical calibration system; and visualization in support of the scientific visualization framework according to the presently disclosed subject matter.

FIG. 15B illustrates an exemplary encoded maternal term with associated elemental labels and numeric values, provided as an additional encoded biological vocabulary example. This figure supports encoded lexical processing in support of the scientific verification system and visualization in support of the scientific visualization framework according to the presently disclosed subject matter.

FIG. 15C illustrates an exemplary family-related encoded composition including parental and child terms with associated numeric values, demonstrating encoded aggregation and output relationships. This figure supports computation and output reporting in support of the scientific verification system and visualization in support of the scientific visualization framework according to the presently disclosed subject matter.

FIG. 16 illustrates an exemplary combined visualization integrating pregnancy, conception, and encoded biological terminology within a single conceptual layout. This figure supports integrated visualization in support of the scientific visualization framework and contextual illustration of developmental benchmarks in support of the scientific verification system and medical calibration system according to the presently disclosed subject matter.

FIG. 17 illustrates an exemplary composite framed visualization combining multiple encoded representations into a unified presentation, provided as an example of an integrated scientific visualization artifact. This figure supports the scientific visualization framework according to the presently disclosed subject matter.

### DETAILED DESCRIPTIONS OF THE EMBODIMENTS/APPLICATIONS

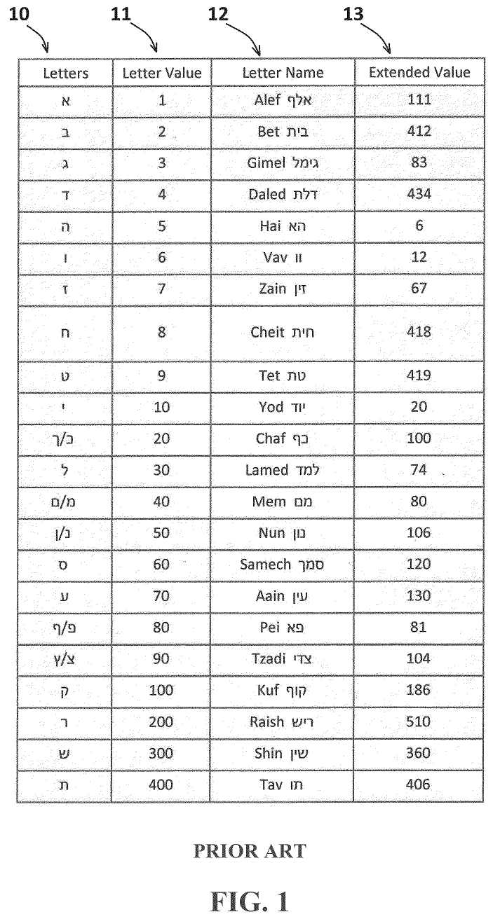

Referring now to the drawings with more specificity, the following specifications generally describe a system and method for deriving secondary or code2GOD meaning from an original Bible-coded letter string comprising a single continuous string of 1,197,000 Hebrew letters devoid of spaces and punctuation. These specifications describe a number of codes or algorithms for deciphering or deriving secondary or code2GOD meaning from the Hebrew bible as exemplified by the original Hebrew bible letter string noting that each Hebrew letter of the Hebrew alphabet has an inherent value associated therewith. In this regard, the reader is firstly directed to FIG. 1 detailing or listing (a) the twenty-two Hebrew letters as set forth in Column 10; (b) the numerical letter values associated with each Hebrew letter as set forth in Column 11; (c) the letter name or nomenclature for each Hebrew letter written in both English and corresponding Hebrew as set forth in Column 12; and (d) extended word values for the Hebrew letter names as set forth in Column 13.

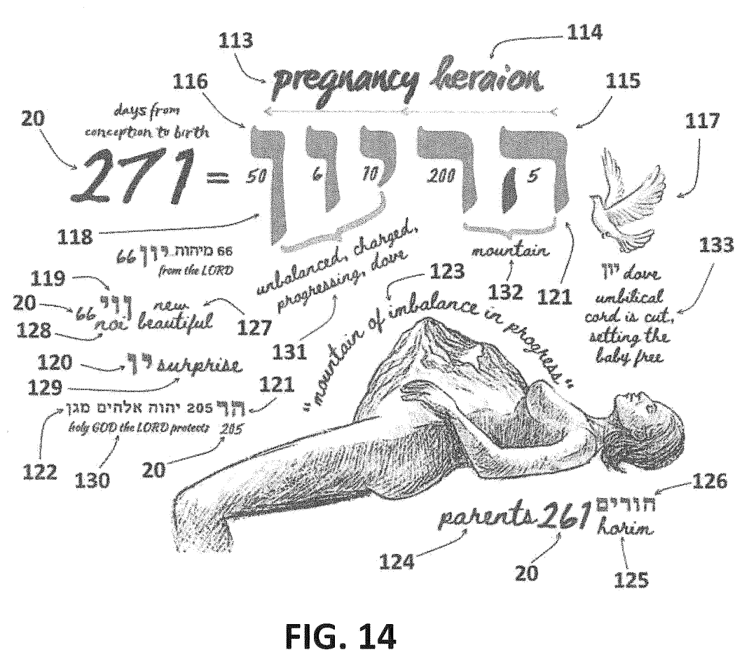

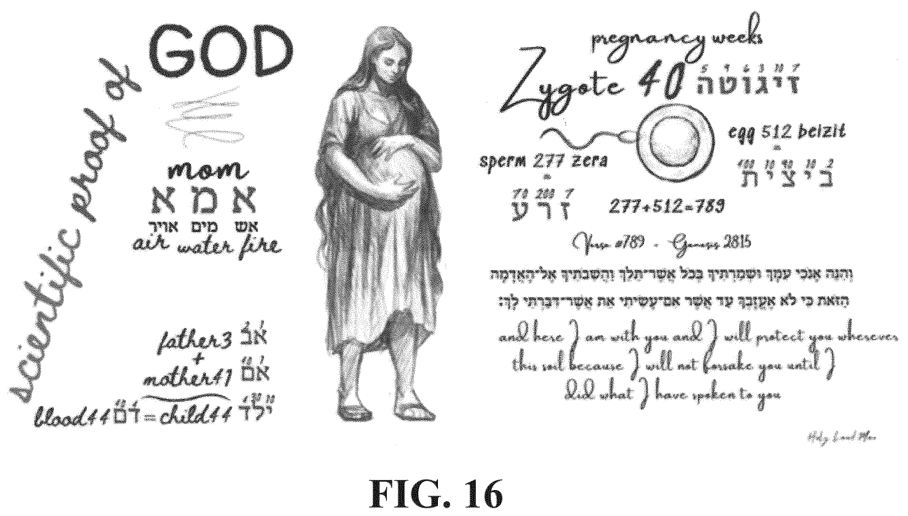

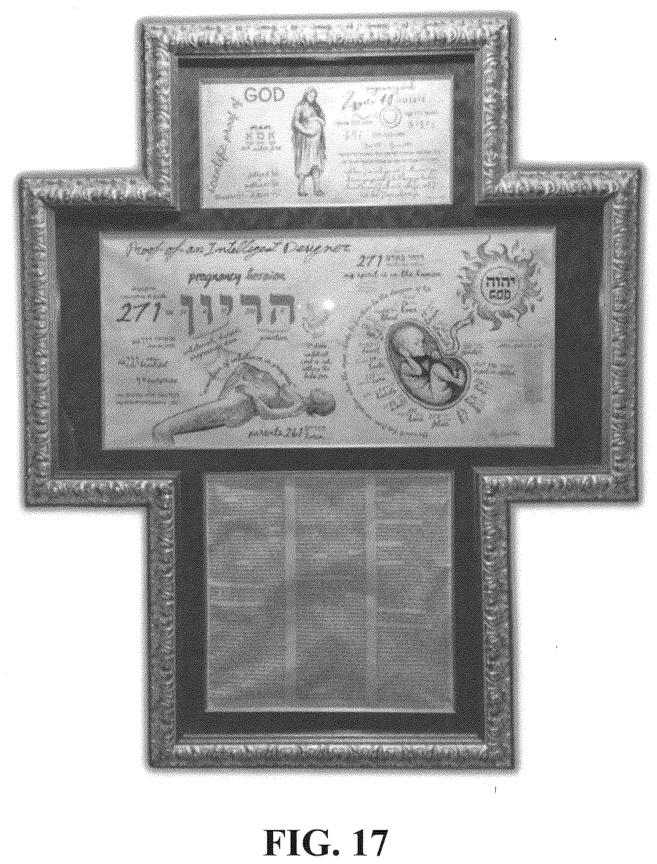

FIGS. 1-12 generally support encoding, computation, and visualization context aspects of the presently disclosed subject matter; FIGS. 13-15C are the core five figures according to the presently disclosed subject matter supporting biological proof (heart, kidney, gestation, conception, and life); and FIGS. 16-17 generally support system integration and a visualization framework. Referencing FIG. 1, the reader will note that Each of the biblical Hebrew letters set forth in Column 10 comprises or is associated with a numerical letter value (letter=value) as further set forth in corresponding rows in Column 11.

While an English letter is only a pronounced symbol, each of the biblical Hebrew letters further corresponds or is associated with a personal name comprising a plurality of Hebrew numerical value-based letters. For ease of understanding both English and Hebrew names of each Hebrew letter are presented or set forth in Column 12. The name or nomenclature of each Hebrew letter has a numerical value called an “extended value” as set forth in Column 13. A first code or algorithm or Code No. 1 upon which the presently disclosed subject matter builds is a biblical numerical letter value of each Hebrew letter. These numerical letter values are set forth in Column 11. A second code or algorithm or Code No. 2 upon which the presently disclosed subject matter builds is the extended word value of each Hebrew letter name set forth in Column 12. These extended word values are set forth in Column 13.

For example in a first instance, the biblical Hebrew letter “ [symbol] ” has a numerical letter value of 1 and is pronounced like the letter, “A” in the English language. The name or nomenclature for the biblical Hebrew letter, “ [symbol] ” is “Alef” in the English language or, “ [symbol] ” in the Hebrew language with an extended word value of 111 as calculated by summing the numerical letter values for each Hebrew letter within the letter name. From left to right for ease of understanding, the Hebrew letter, “ [symbol] ” is associated with the numerical letter value 80, the Hebrew letter, “ [symbol] ” is associated with the numerical letter value 30; and the Hebrew letter, “ [symbol] ” is associated with the numerical letter value 1.

These numerical letter values sum to a word value (20) of 111 and this total is the extended word value for the named Hebrew letter, “ [symbol] ”. For example in a second instance, the biblical Hebrew letter, “ [symbol] ” letter value is 100, pronounced like, “K” in the English language. Its English name is, “Kuf” and its Hebrew name is, “ [symbol] ” with an extended word value of 186. From left to right for ease of understanding, the Hebrew letter, “ [symbol] ” is associated with the numerical letter value 80, the Hebrew letter, “ [symbol] ” is associated with the numerical letter value 6; and the Hebrew letter, “ [symbol] ” is associated with the numerical letter value 100. These numerical letter values sum to 186 and this total is the extended word value for the named Hebrew letter, “ [symbol] ”.

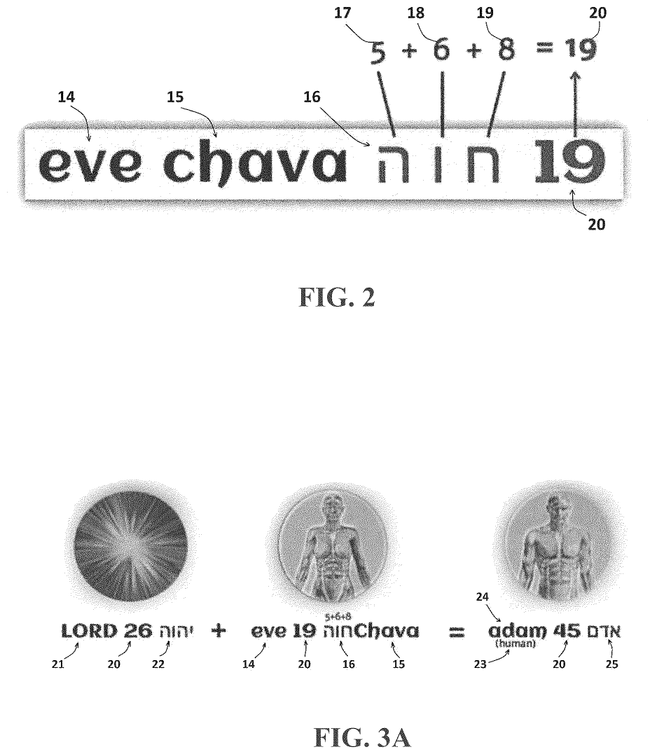

A third code or algorithm or Code No. 3 upon which the presently disclosed subject matter builds is a word value (20) associated with a Hebrew word. In this regard, a Hebrew word comprises a series of Hebrew letters and the numerical letter values of each Hebrew letter within the Hebrew word are added to provide the word value (20). In this regard, the reader is directed to FIG. 2, which figure provides an example of how a word value (20) is calculated for the exemplary English word, “Eve” as at (14) otherwise pronounced as “Chava” as at (15) in Hebrew and written as, “ [symbol] ” as at (16) in Hebrew. From left to right for ease of understanding, the Hebrew letter, “ [symbol] ” is associated with the numerical letter value 5 as at (17); the Hebrew letter, “ [symbol] ” is associated with the numerical letter value 6 as at (18); and the Hebrew letter, “ [symbol] ” is associated with the numerical letter value 8 as at (19). These numerical letter values sum to 19 and this total is the extended word value (20) for the Hebrew word, “ [symbol] ” (16).

Word values (20) may essentially be used anagrammatically or in equations to yield code2GOD messaging or secondary meaning as derived from the primary meaning of the words within the original Hebrew biblical letter string. Secondary or code2GOD meaning derived from the original Hebrew biblical letter string may relate to how the human environment is structured and how humans are wired for a meaningful life within that human environment. These code2GOD messages or the secondary meaning associated therewith may be regarded as scientific, social, and meant to improve the wellbeing of humanity. This secondary or code2GOD meaning can help bolster one's appreciation and understanding of environmental structure and the role humans play in that environmental structure.

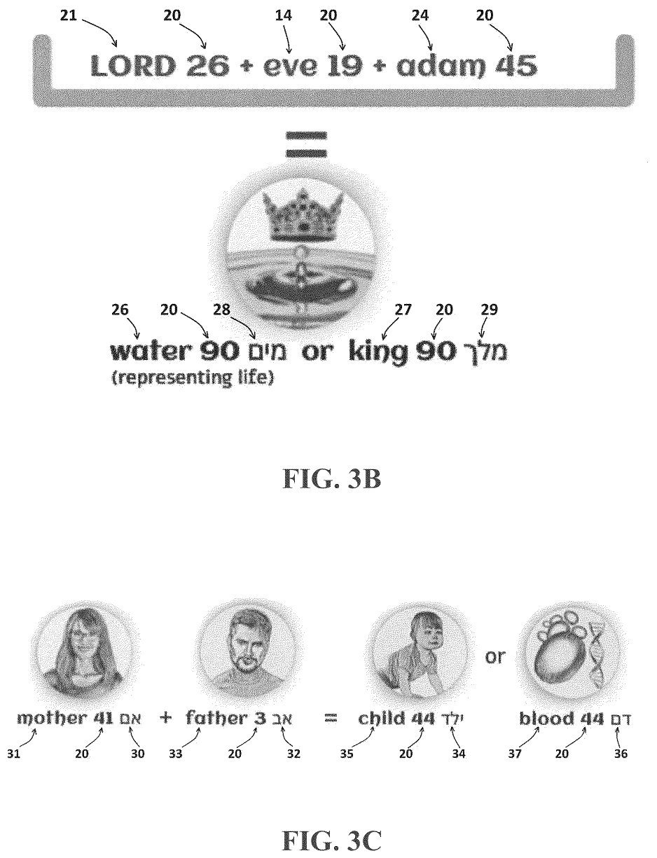

A fourth code or algorithm or Code No. 4 according to the presently disclosed subject matter is the use of Hebrew words as meaningful equations, and is illustrated by the following examples as further supported by FIGS. 3A-6. A first example of the use of Hebrew words as equations or Code No. 4 according to the presently disclosed subject matter is generally illustrated in FIG. 3A in the form of a first simple addition operation. Referencing FIG. 3A, the reader will there first consider the English word, “Lord” as at (21) otherwise written as, “ [symbol] ” in Hebrew as at (22). The word value (20) for, “Lord” (21) or, “ [symbol] ” (22) is 26 in Hebrew. The reader will further recall the word value (20) for the English word, “Eve” (14) or “Chava” (15) or “ [symbol] ” (16) in Hebrew is 19. Summing the word values (20) of 26 for, “Lord” (21) or “ [symbol] ” (22) and 19 for “Eve” (14) or “Chava” (15) or “ [symbol] ” (16) equals or provides a word value (20) of 45. The word value (20) of 45 equates with the word value (20) of the Hebrew word, “ [symbol] ” as at (25) otherwise written in English as “human” as at (23) or “Adam” as at (24).

Referencing FIG. 3B, the reader will there consider a second example of the use of Hebrew words as equations or Code No. 4 according to the presently disclosed subject matter in the form of a second simple addition operation. Recalling the word value (20) for the Hebrew word or term, “ [symbol] ” (22) (or in English, “Lord” (21)) equals 26 and that the word value (20) for “Chava” (15) or “ [symbol] ” (16) in Hebrew (or in English, “Eve” (14)) is 19 the decoder can then add the Hebrew word, “ [symbol] ” (25) with a word value (20) of 19. The total resulting word value (20) is 90, which corresponds to the word value (20) for either, “ [symbol] ” (28) or “ [symbol] ” (29) in Hebrew which translate to “water” (26) (representing life) or “king” (27) in English, respectively.

Referencing FIG. 3C, the reader will there consider a third example of the use of Hebrew words within simple word-based equations or Code No. 4 according to the presently disclosed subject matter in the form of a third simple addition operation. The word value (20) for the Hebrew term, “ [symbol] ” (22) (or in English, “mother” (31)) equals 41 and the word value (20) for the Hebrew term, “ [symbol] ” (32) (or in English, “father” (33)) equals 3. Adding the word values (20) of “ [symbol] ” or 41 and “ [symbol] ” or 3 equals a word value (20) of 44, which word value (20) corresponds to either the Hebrew term, “ [symbol] ” or in English, “child” (35), or the Hebrew term, “ [symbol] ” (36) or in English, “blood” (37). When interpreted by a chosen interpreter, the resulting secondary or code2GOD meaning may translate into a message that a “mother” (31) and a “father” (33) together yield a “child” (35) or alternatively, “blood” line (37).

Referencing FIG. 3D, the reader will there consider a fourth example of the use of Hebrew words as equations or Code No. 4 according to the presently disclosed subject matter in the form of a simple subtraction operation. The word value (20) for the Hebrew term “ [symbol] ” (38) (or in English, “desired” (39)) equals 306 and that the word value (20) for the Hebrew term, “ [symbol] ” (40) (or in English, “available” (441)) equals 146. Subtracting the word value (20) of the Hebrew “ [symbol] ” (40) or 146 from the Hebrew “ [symbol] ” (38) or 306 equals a word value (20) of 160, which word value (20) corresponds to the Hebrew term, “ [symbol] ” (42) (or in English, “money” (43)). When interpreted by a chosen interpreter, the resulting secondary or code2GOD meaning may translate into a message that “money” (43) can close the gap between the “desired” (39) and the “available” (41). A fifth non-illustrated example of the use of Hebrew words as equations or Code No. 4 according to the presently disclosed subject matter in the form of a second simple subtraction operation, as follows: Male=227=zachar=“ [symbol] ” minus Female=157=nekeva=“ [symbol] ”=Confusion=70=bilbul=“ [symbol] ”.

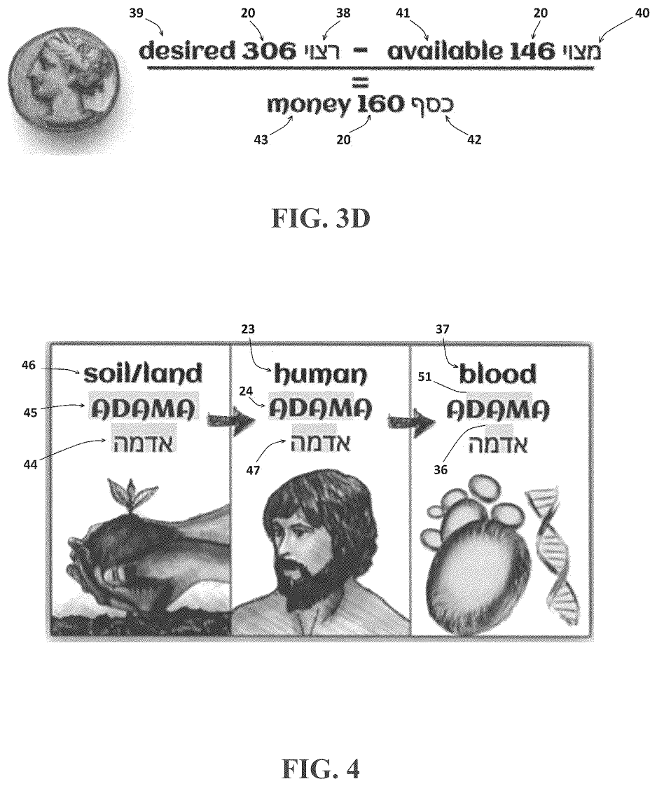

Referencing FIG. 4, the reader will there consider a first example of a fifth code or algorithm or Code No. 5 according to the presently disclosed subject matter. Code No. 5 is essentially the telling of a story in a single Hebrew word. The Hebrew letters that make up the Hebrew word can be reassembled in a different order or broken into sub-words to create a message or a story, while maintaining the same overall word value. For example, the Hebrew word, “ [symbol] ” (44) or in English “Adama” (45) (meaning land/soil (46)) having a word value (20) of 50 comprises the Hebrew word, “ [symbol] ” (47) or in English Adam (24) meaning “human” (23) having a word value (20) of 49, which Hebrew word, “ [symbol] ” (47) further comprises the Hebrew word, “ [symbol] ” (36) or in English “dam” (51) meaning blood (37) with a word value (20) of 44. The resulting secondary or code2GOD meaning may translate into a message that humans have a soul-blood bond with the land or that humans were originated from and shall return to buried in/become soil.

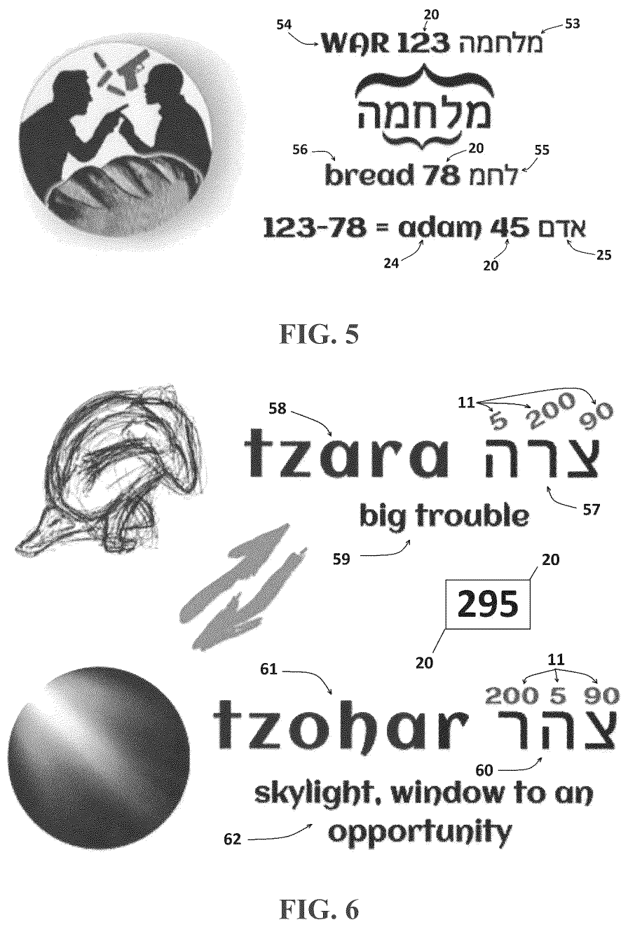

Referencing FIG. 5, the reader will there further consider a second example of Code No. 5 according to the presently disclosed subject matter. The Hebrew word, “ [symbol] ” (53) or in English “war” (54) having a word value (20) of 123 comprises the Hebrew word, “ [symbol] ” (55) or in English “bread” (56) having a word value (20) of 78. The word value (20) of 123 for “war” (54) less the word value (20) of 78 for “bread” (56) yields a word value (20) of 45, which word value (20) 45 corresponds to or equates with the word value (20) of the Hebrew word, “ [symbol] ” (25) otherwise written in English as “human” (23) or “Adam” (24). The resulting secondary or code2GOD meaning may translate into a message that humans were created to always fight for their bread or existence.

Third and fourth examples of Code No. 5 not specifically illustrated include the following: the Hebrew word, “ [symbol] ” or “kelev” meaning “dog” in English has a word value (20) of 52. By subtracting, “ [symbol] ” with word value (20) of 20 from, “ [symbol] ” with word value (20) of 52, the result is, “ [symbol] ” with word value (20) of 32 with English translation, “like a heart”. Further, the six Hebrew letters that together form the first Hebrew word of the original Bible are, “ [symbol] ” or “bereshit” meaning “Genesis” in English and has a combined word value (20) of 913. These individual Hebrew letters can be rearranged into at least twenty-two separate Hebrew words, each with a related meaning to the concept of creation. The Chosen interpreter may then utilize these twenty-two anagram words to better elaborate upon or otherwise explain secondary or code2GOD meaning relating to the formation of the universe.

A sixth code or algorithm or Code No. 6 according to the presently disclosed subject matter relates to Hebrew words having identical word values (20). In other words, different Hebrew words that share the same word value (20) can also be interpreted to share other attributes in common for revealing secondary or code2GOD meaning as exemplified by the concepts of love, money, family, soul, life purpose, etc. For example, a word value (20) of 13 may be equated with the following: love or “ [symbol] ”; one or “ [symbol] ”; fairytale or “ [symbol] ”; worry or “ [symbol] ”; and lost or “ [symbol] ”. A word value (20) of 160 may be associated with money or “ [symbol] ”; tree or “ [symbol] ”; codes or “ [symbol] ”; and tone or “ [symbol] ”. A word value of 395 may be equated with higher soul or “ [symbol] ”; and the skies or “ [symbol] ”. A word value of 90 may be associated with water or “ [symbol] ” and newborn or “ [symbol] ”. A word value of 160 may be equated with tree or “etz” or “ [symbol] ”=money or “kesef” or “ [symbol] ”=codes or “kodim” or “kesef” or “ [symbol] ” The resulting secondary or code2GOD meaning may translate into a message that having the right codes (like formula) can help one grow more greens (money) and branches.

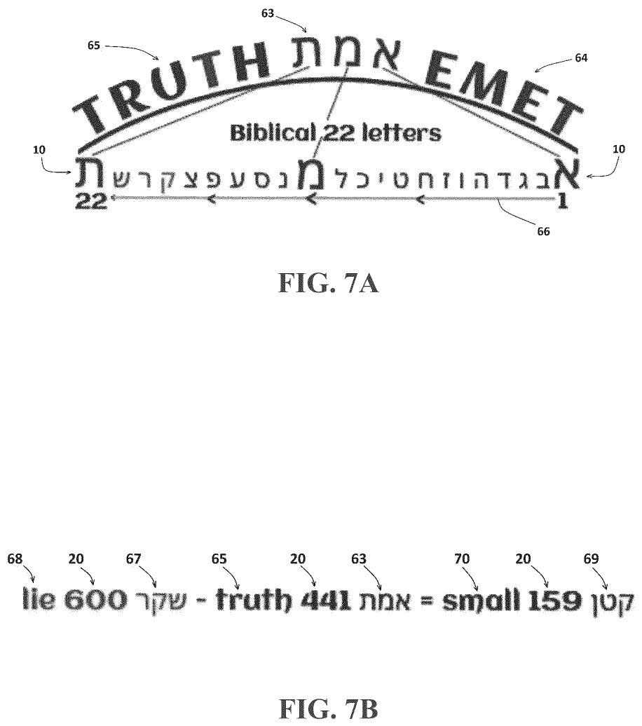

A seventh code or algorithm or Code No. 7 according to the presently disclosed subject matter involves the provision of parable messaging via anagram. In other words, the rearrangement of Hebrew letters (each having a numerical letter value (11)) within a Hebrew word yields a different secondary or code2GOD meaning while maintaining the same numerical word value (20). Referencing FIG. 6, the reader will there consider the Hebrew word, “ [symbol] ” (57) or “tzara” (58) or in English “big trouble” (59). This Hebrew word has a word value (20) of 295. The anagram “ [symbol] ” (60) or “tzohar” (61) or in English “skylight or window to an opportunity” (62) comprises the same word value (20) of 295.

The resulting secondary or code2GOD meaning may translate into a message that trouble (with an inherent negative connotation) may provide a window to an opportunity (with an inherent positive connotation). In other words, when encountering “big trouble” (59) or “ [symbol] ” (57) or “tzara” (58), look for the “bright opportunity” (62) or “ [symbol] ” (60) or “tzohar” (61). Conversely, when encountering “ [symbol] ” (60) or “tzohar” (61) or an “opportunity” (62), be careful to avoid “ [symbol] ” (57) or “tzara” (58) or “big trouble” (59). Other non-illustrated examples include a word value (20) of 78 which equates with either “ [symbol] ” or in English “bread” or “ [symbol] ” or in English “salt”; and the word value (20) of “ [symbol] ” or “yekum” or in English universe or “ [symbol] ” or “ki-iom” or in English “survival”.

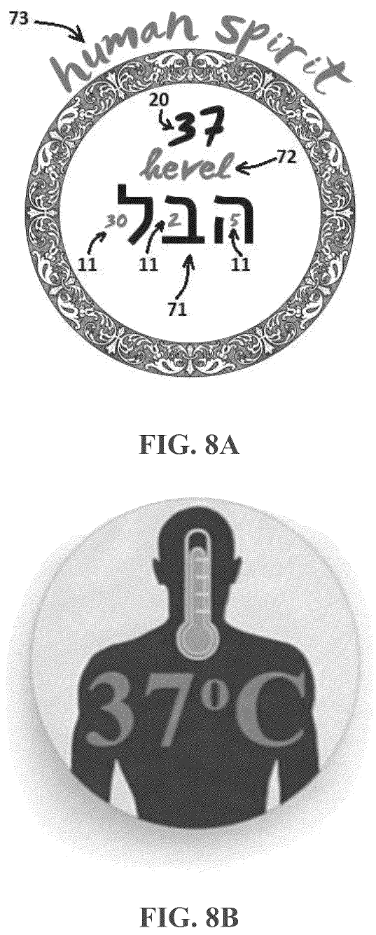

An eighth code or algorithm or Code No. 8 according to the presently disclosed subject matter involves the position of the respective Hebrew letters within the table otherwise presented in FIG. 1. In other words, observing the letters' relative (or absolute) position on the lineup from the first Hebrew letter, “ [symbol] ” to the last Hebrew letter, “ [symbol] ”, can be seen to reveal secondary or code2GOD meaning. In this regard, the reader is directed to FIG. 7. Referencing FIG. 7, the reader will there consider the Hebrew word, “ [symbol] ” (63) or “emet” (64) or in English, “truth” (65). The Hebrew word, “ [symbol] ” (63) comprises the first Hebrew letter, “ [symbol] ” with a numerical letter value of 1; the last Hebrew letter, “ [symbol] ” with a numerical letter value of 400; and a middle Hebrew letter, “ [symbol] ” with a numerical letter value (11) of 40 combining to provide a total word value (20) of 441. The resulting secondary or code2GOD meaning may translate into a message that “truth” (65) is not definite and can be found within the entire spectrum of possibilities as at (66).

Comparatively referencing FIGS. 7A and 7B, the reader will further consider the Hebrew word, “ [symbol] ” (67) or “sheker” or in English, “lie” (68). The Hebrew word, “ [symbol] ” (67) comprises the 19^{th}, 20^{th}, and 21^{st} Hebrew letters ahead of the last Hebrew letter, “ [symbol] ”. Noting that the last or 22^{nd} Hebrew letter, “ [symbol] ” is found within the Hebrew word, “ [symbol] ” (63) for “truth”, a secondary or code2GOD meaning that may be derived from the Hebrew word, “ [symbol] ” (67) is that humans are wired to know, or identify, what a “lie” (68) is, which is also intentional, according to Code No. 8. However, even a “lie” (68) is set within a slim possibility of being within the spectrum of the truth. Furthermore, humans should not shrink from truth telling and for the decoder/student to be open to receiving the truth on the entire spectrum of life's possibilities. Recalling Code No. 4, (Words As Meaningful Equations) the reader will further see that the difference between a “lie” (68) with a word value (20) of 600 and the “truth” (65) with a word value (20) of 441 can be “small” (70) or in Hebrew, “ [symbol] ” which Hebrew term equates to a total word value (20) of 159.

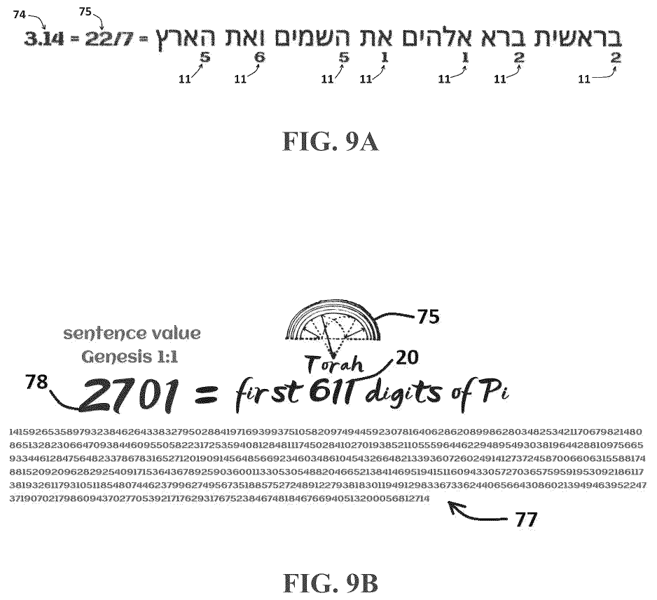

A ninth code or algorithm or Code No. 9 according to the presently disclosed subject matter involves the identification of a mystery, dominant, meaningful, or repetitive number within the numerical word values (20) provided by the Hebrew language. In this regard, the chosen interpreter will cross reference word values (20) with facts of life to provide code2GOD messaging or secondary meaning as derived from these mystery, dominant, meaningful, or repetitive number values inherent to the Hebrew language. FIGS. 8A through 8C are presented in support of Code No. 9. Referencing FIG. 8A, the reader will there consider the first verse of the original Hebrew bible comprises a word value (20) sum of 2701 as derived from the addition of all 28 letters of this first verse.

An example of a mystery, dominant, meaningful, or repetitive number within the numerical word values (20) provided by the Hebrew language is the number 37. The reader will note that the number 2701 as derived by the addition of all 28 Hebrew letters of the first verse of the original Hebrew bible is also the product of the numerical value 37 multiplied by the numerical value 73. The number 2701 is also the sum of 37+999+888+777. The number 37 is also a multiplier of 999 (i.e. 37×27), of 888 (i.e. 37×24) and of 777 (i.e. 37×21). According to the presently disclosed subject matter, the chosen interpreter may derive the importance of the number 37 and relate to this to the human condition as derived from within the Hebrew language. The number 37, for example, is the human body temperature (and also the body temperature of certain mammals: whale (sea), bat (air) and lion (ground)) in Celsius degrees as generally depicted in FIG. 8B, and may thus be translated as evidencing the “human spirit” (73) as comparatively referenced in FIG. 8A. Referencing FIG. 8A, the reader will there consider the word value (20) of the Hebrew word, “ [symbol] ” (71) or “Hevel” (72) comprising the first Hebrew letter, “ [symbol] ” and the last Hebrew letter, “ [symbol] ” of the Torah and the original Bible and in English is “Habel” (Hevel or [symbol] ) representing the “human spirit” (73).

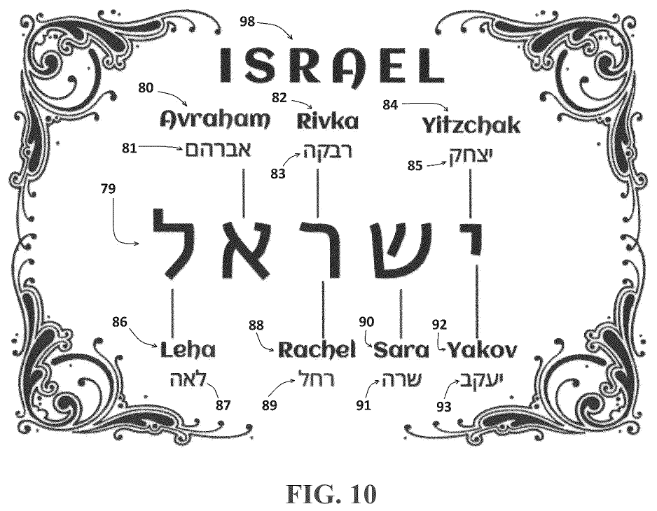

A tenth code or algorithm or Code No. 10 according to the presently disclosed subject matter involves the use of pi or in Greek, “ [symbol] ”. It is noted that the numerical value of pi (the ratio of a circle's circumference to its diameter) is commonly understood to comprise an infinite number of digits after the primary integer value of 3 as in 3.14159 . . . . The concept of pi or “ [symbol] ” is encoded in the first verse of the original Bible in the Hebrew language. In this regard, it is noted that the value of 3.14 (74) is the average letter value of each of the first letters of each of the seven words of the first verse of the original Bible in Hebrew. In this regard, the reader is directed to FIG. 9A. Referencing FIG. 9A, the reader will there consider the first seven Hebrew words of the first verse with numerical letter values (11) presented for each of the first seven Hebrew words. Summing these numerical letter values provides a sum of 22 as at (75), which when divided by the number of Hebrew words or 7 yields 3.14 (74). This so-called pi decoder or π decoder feature aids in deriving secondary or code2GOD meaning or verifying previous findings. Code No. 9 according to the presently disclosed subject matter utilizes four pi/π parameters, namely, (1) the sum of a set string of numbers; (2) the number of digits in a set string; (3) the starting digit number in the pi/π infinite string; and (4) adjacent coded set of numbers.

For example, referencing FIG. 9B, the reader will there consider a representation of the first 5 books of the Bible commonly referred to as the Torah as at (76) or in Hebrew, “ [symbol] ” with a word value (20) of 611. The first 611 post decimal digits as at (77) of pi/π sum to the numerical value of 2701 as at (78), which numerical value 2701 (78) is exactly the verse value of biblical Genesis 1:1 describing the concept of creation. The resulting secondary or code2GOD meaning may translate into a message that the world was created based on the Torah (76) of the Bible, and that the pi/π code of Code No. 9 may help function as a cross-reference and/or confirmation of the primary meaning otherwise derived from the face value of the first verse and the Hebrew word, “ [symbol] ”.

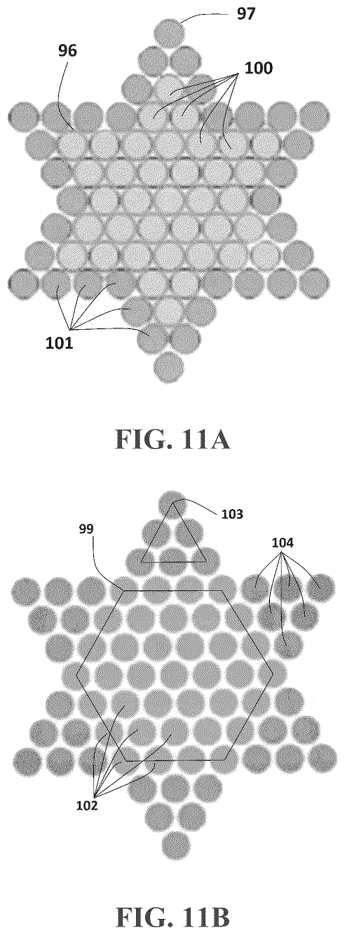

An eleventh code or algorithm or Code No. 11 according to the presently disclosed subject matter involves the use of acronyms to form word values (20) for the purpose of deriving secondary or code2GOD meaning from the Hebrew words from which initial Hebrew letters are lifted to form the acronym. In this regard, it is noted that an acronym is an abbreviation formed from the initial letters of other words and pronounced as a word. Referencing FIG. 10, the reader will there consider the first Hebrew letter of each of three fore fathers' and the four fore mothers' names make up the acronym name, “Israel” (98) or in Hebrew, “ [symbol] ” (79). The three fore father' names are “Avraham” (80) or in Hebrew, “ [symbol] ” (81); “Rivka” (82) or in Hebrew, “ [symbol] ” (83), and “Yitzchak” (84) or in Hebrew, “ [symbol] ” (85). The four fore mothers' names are “Leha” (86) or in Hebrew, “ [symbol] ” (87); “Rachel” (88) or in Hebrew, “ [symbol] ” (89); “Sara” (90) or in Hebrew, “ [symbol] ” (91); and “Yakov” (92) or in Hebrew, “ [symbol] ” (93). A secondary or code2GOD meaning derived from the acronym name, “Israel” (98) or in Hebrew, “ [symbol] ” (79) is that the for elders are inherently tied to the state or concept of Israel.

A twelfth code or algorithm or Code No. 12 according to the presently disclosed subject matter involves the use of natural logarithm to derive secondary or code2GOD meaning from word values (20). The natural logarithm of a number is its logarithm to the base of the mathematical constant e, which is an irrational and transcendental number approximately equal to 2.718281828459. Code No. 12 is used to calculate the scientific correlation of spatial information such as distances (e.g. the distance from the Earth to the sun); temporal information such as periods of time; and other perceptive information including audible information (e.g. sounds), visual information (e.g. colors), frequency information, etc.

A thirteenth code or algorithm or Code No. 13 according to the presently disclosed subject matter involves the use of simplified numerology. Given a word value (20), the single digits of the word value (20) may be summed to arrive a final reduced number between 1 and 9. If the finally reduced number is 11, 22, 33, or 44, these numbers will not be reduced any further for they are considered unique numbers. For example a word value (20) of 728 is firstly reduced to 17 by adding 7, 2, and 8 or 7+2+8=17, and then secondly reducing 17 or 1+7=8. The word value (20) of 911 finally reduces to 11 and not 2 since 11 is one of the four unique numbers.

Recalling the mystery, dominant, meaningful, or repetitive number 37 (e.g. body temperature is 37° C.), it will be seen the number firstly reduces to 10 (perfection) and secondly reduces to 1. A secondary or code2GOD meaning that may be derived from the number 37 is that the firstly reduced number 10 may mean, “perfection” and the secondly reduced number of 1 may mean, “new beginnings or the concept of divinity”. A further example is illustrated by the simple numerology or finally reduced number that can be used to show that various words having the same finally reduced number have something in common. For example, the word, “lung” or in Hebrew, “ [symbol] ” has a word value (20) of 180; the word, “nose” or in Hebrew, “ [symbol] ” a word value (20) of 81; the word, “nature” or in Hebrew “ [symbol] ” has a word value (20) of 81; the word, “face” or in Hebrew, “ [symbol] ” has a word value (20) of 180; and the word, “eyes” or in Hebrew, “ [symbol] ” has a word value of 180. All of these word values (20) finally reduce to the number 9 and all the words have anatomical connotations in common.

A fourteenth code or algorithm or Code No. 14 according to the presently disclosed subject matter involves interval skipping within a string of Hebrew letters. There are three factors to consider when finding a Hebrew word within a string of Hebrew letters as exemplified by the original Bible letter string comprising 1,197,000 Hebrew letters. These three factors include (1) the starting or initial Hebrew letter in the string, (2) the number of Hebrew letters to skip over, and (3) directional movement within the string (i.e. forward or backward). The reader will here consider the Hebrew word, “ [symbol] ” or “malachim” or in English, “Angels” having a word value (20) of 141. This word can be derived from biblical Genesis 1:8 as at (94) starting with the initial letter number 663 and interval skipping in a backward direction a total of 6 letters until the Hebrew word, “ [symbol] ” is finally formed.

Note that the word value (20) of 141 finally reduced to simple numerology is 6 which is the number of interval skipped letters in this example. It is estimated that 0.1% of human DNA is unique to every human, which translates into roughly 32 million repetitions of the compounds Adenine (A), Cytosine (C), Guanine (G) and Thymine (T). The original Hebrew Bible string comprises 1,197,000 Hebrew letters of which there are 22 in the Hebrew alphabet or Alef-Beit. It is noted that locating genetic traits within human DNA is similar to finding meaningful words and events within the original Hebrew Biblical string and given the complexity of such sequences may be preferably performed by a computer.

A fifteenth code or algorithm or Code No. 15 according to the presently disclosed subject matter follows from the observation that a set of Hebrew words can be correlated for deriving secondary or code2GOD meaning therefrom by way of applying mathematical operations to a given Hebrew word and its corresponding word value (20). For example, multiplications of the word, “lord” (21) or in Hebrew, “ [symbol] ” or “yhvh” (22) having a word value (20) of 26 may yield word value (20) multiples of 26 and provide the chosen interpreter with information relating to the given word.

A further example is illustrated by the Hebrew word, “ [symbol] ” (81) or “Avraham” (80) or in English, “Abraham” having a word value (20) of 248. The Hebrew word, “ [symbol] ” or in English, “eden” has a word value (20) of 124 and multiplied by 2 equates to 248 as in 124×2=248. Eden may thus correlate with Abraham via a multiplier of 2 and this information can be used to derive secondary or code2GOD meaning from the words illustrated. Further, the Hebrew names, “ [symbol] ” or “Yitzchak” or in English, “Isaac”; “ [symbol] ” or “Yakov” or in English, “Jacob”; and “ [symbol] ” or “Yosef” or in English, “Josef” have corresponding word values (20) of 208=26×8; 182=26×7; and 156=26×6, respectively. The common multiplier of 26 is found in each word value (20) for these Hebrew names and this information can be used by the chosen interpreter to derive secondary or code2GOD meaning from the Hebrew names.

Recalling the original Bible's first verse sum of all 28 letters is 2701 as at (78), the reader will note that this number is the product of the dominant, meaningful, or repetitive number 37 multiplied by 73 as in 2701=37×73. The number 2701 (78) is also the sum of 37+999+888+777. The dominant, meaningful, or repetitive number 37 number 37 is also a factor of 999 (as in 999=37×27), of 888 (as in 888=37×24), and 777 (as in 777=37×21). A secondary or code2GOD meaning that can be derived from the numbers 2701 and 37. As previously described, 37 degrees Celsius is the average human body temperature and that of certain mammals including whales (with sea habitat), bats (with air habitat) and lions (with ground habitat). The number 37 is also the word value (20) of “Hevel” or “ [symbol] ” comprising the first letter ( [symbol] ) and the last letter ( [symbol] ) of the Torah (75) and the original Bible. The number 37 is also the word value (20) corresponding to the Hebrew, “ [symbol] ” (71) or “Hevel” (72) or in English, Abel representing the human spirit (73).

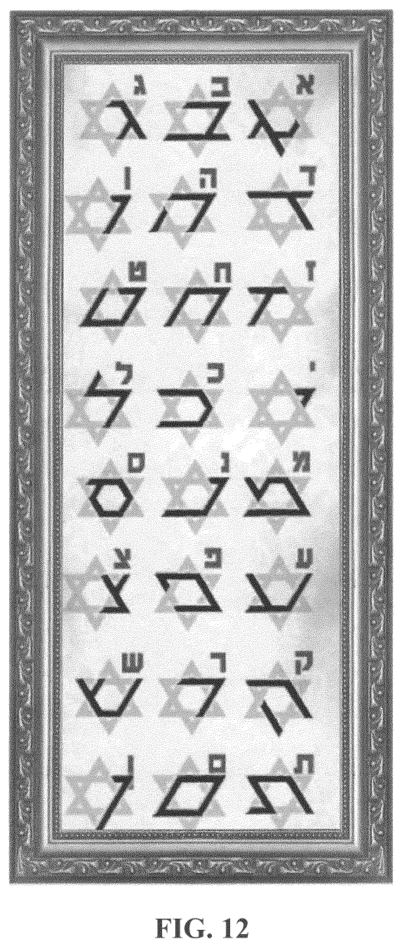

A sixteenth code or algorithm or Code No. 16 according to the presently disclosed subject matter involves the use of geometrical shapes from which to derive secondary or code2GOD meaning. The Star of David Theorem is a mathematical result on arithmetic properties of binomial coefficients. Comparatively referencing FIGS. 11A and 11B it will there be seen that the numbers 37 and 73 are inherently represented by the Star of David theorem. FIG. 11A comprises an inner hexagram as at (96) comprising 37 points (100) shown in first or relatively light grayscale coloration peripherally bound an outer hexagram as at (97) comprising 36 points (101) shown in second or relatively dark grayscale coloration together summing to a total of 73 points.

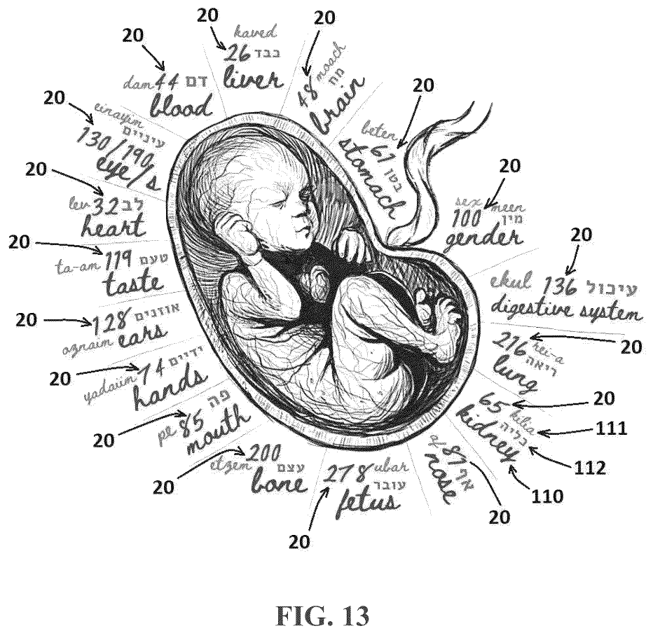

FIG. 11B by contrast comprises an inner hexagon (99) comprising 37 points (102) with 6 peripheral triangles (103) each of which comprise 6 points (104) summing to a total of 73 points. As previously described, the number 2701 (78) is the product of 37 and 73 as found within the original Hebrew bible string. The Star of David may also comprise a total of 541 points which equates with the word value (20) for the Hebrew, “ [symbol] ” (79) or in English, “Israel” (98). Referencing FIG. 12C it will be seen that the Hebrew letter forms of which there are 24=22+2 all correspond to portions of the Star of David. It will thus be seen that geometrical shape information can also be used to convey secondary or code2GOD meaning as derived from the original Hebrew bible letter string.

A seventeenth code or algorithm or Code No. 17 according to the presently disclosed subject matter involves counting the number of letters in a word, related words, or a sentence. For example, there are 13 Hebrew letters in the four names of the fore mothers: “Leha” (86) or in Hebrew, “ [symbol] ” (87); “Rachel” (88) or in Hebrew, “ [symbol] ” (89); “Sara” (90) or in Hebrew, “ [symbol] ” (91); and “Yakov” (92) or in Hebrew, “ [symbol] ” (93). Further, there are 13 Hebrew letters in the three names of the fore fathers: “Avraham” (80) or in Hebrew, “ [symbol] ” (81); “Rivka” (82) or in Hebrew, “ [symbol] ” (83), and “Yitzchak” (84) or in Hebrew, “ [symbol] ” (85). The number 13 is the word value (20) for the Hebrew word, “ [symbol] ” or in English, “love”. Adding the number 13 to 13 yields 26, which is the word value (20) for the Hebrew word, “ [symbol] ” (22) or in English, “Lord” (21). These numerical results point to specific verse numbers between 1 and 23,204.

Similarly, the number of singular word repetitions in the original Hebrew Bible string can provide meaningful word values (20). Further, matching the first and last letters of a Hebrew word with the first and last letters of a verse from the original Hebrew bible string provides information for deriving additional secondary or code2GOD meaning. Further, matching name word value (20) with verse value or consecutive word value provides information for deriving additional secondary or code2GOD meaning. Further, using prime numbers to explain correlations between Hebrew words or verses provides information for deriving additional secondary or code2GOD meaning. Still further, using sequential Fibonacci numbers as reference for related Hebrew words provides information for deriving additional secondary or code2GOD meaning.

An eighteenth code or algorithm or Code No. 18 according to the presently disclosed subject matter involves observation of various characteristics of each Hebrew letter to derive secondary or code2GOD meaning. In this regard, it is noted that each Hebrew letter comprises a particular form, a name, and a name value. In this regard, the particular form of Hebrew letters can be analyzed for providing secondary or code2GOD meaning. The number of termination points of each Hebrew letter; the directional position of termination points relative to the four primary directions (i.e. up, down, right, and left); the relative size of individual Hebrew letters compared to one another; and the relative position of individual Hebrew letters within the formal Hebrew alphabet or Alef-Beit provide information for deriving additional secondary or code2GOD meaning. Further, the vibration and fundamental frequency signatures that are inherent to spoken Hebrew letters can be visualized utilizing computer means, which can then be used to derive additional secondary or code2GOD meaning.

Recalling foundational Code No. 1 and foundational Code No. 2 according to the presently disclosed subject matter, it will be seen that word values (20) can be provided for various gestational maturity descriptors or words found within the original Hebrew bible letter string. By comparing these word values (20) to reliable secular gestational maturity information for various organs and developments of the human fetus as it gestationally develops, the decoder can evidence intelligent design of human life by an intelligent designer or God as the author of the original Hebrew bible letter string. The presently disclosed subject matter provides a method for evidencing intelligent design of human life as determined by gestational maturity information as derived from an original Hebrew bible letter string.

Noting that the code2GOD methods according to the presently disclosed subject matter derive results directly from the original Hebrew language as written from an intelligent designer source, true gestational maturity information is manifested, which gestational maturity is characterized by the period of time from the day of conception (zygote formation) to birth. Using the code2GOD methods, the decoder may show the intelligent designer of human life indicates the exact day of organ function maturity following the time of conception. It is noted that the medical community typically relies solely upon on the concept of pregnancy, the length of which is only a best guess prediction and can be inaccurate by as much as 18 days depending on the mother's cycle, delays in ovulation, emotional stress, etc.

To properly calculate pregnancy duration, 14 days or 2 weeks are added to time estimates beginning on the first day of the last menstrual period (LMP) provided by the mother as based on her best recollection. The real ovulation and the conception time could occur 3-4 days earlier or later than that estimated by the medical community. To compensate for the lag in time, the established medical community adds 2 weeks or 14 days to each exact organ maturity date otherwise governed by the intelligent designer or God. The medical community can only state the week range when an organ appears on an ultrasound screen for many of the organs.

The code2GOD methodology according to the presently disclosed subject matter matches gestational periods of time as revealed to a decoder and based on when the designer of life or intelligent designer determined the organ to be functional, hereinafter referred to as “development maturity.” The code2GOD method according to the presently disclosed subject matter comprises the steps of firstly identifying a primary sequence of Hebrew letters, each Hebrew letter within the primary sequence of Hebrew letters being associated with a select numerical letter value. The Hebrew letters within the primary sequence of Hebrew letters are preferably arranged to form the original Hebrew bible letter string comprising 1,197,000 Hebrew letters of which there are 22 separate Hebrew letters as set forth or depicted in FIG. 1.

The method further preferably comprises the step of identifying at least one gestational maturity descriptor within the original Hebrew bible letter string. Referring to FIG. 1, an exemplary embodiment of a scientific verification system is illustrated, showing system components for receiving encoded linguistic input, performing numeric decoding, correlating fixed developmental benchmarks, evaluating statistical non-coincidence, validating invariance, and generating a design-verification output. Considering FIG. 13, the reader will consider the English gestational maturity descriptor, “kidney” as at (110). The English descriptor, “kidney” corresponds to the Hebrew descriptor, “kilia” as at (111) or “ [symbol] ” as at (112), which Hebrew gestational maturity descriptor has a word value (20) equal to 65. The word value (20) of “ [symbol] ” equals the sum of the numerical letter values for each Hebrew letter in the gestational maturity descriptor as in the following: “ [symbol] ” with letter value (11) of 20+“ [symbol] ” with letter value (11) of 30+“′” with letter value (11) of 10+“ [symbol] ” with letter value (11) of 5=65.

Once the word value (20) for a given gestational maturity descriptor is provided, the word value (20) is then divided by a factor of seven to yield a basic or first number of weeks value for the gestational maturity descriptor. In the case of the gestational maturity descriptor example, “kidney” with word value (20) of 65, the word value (20) of 65 divided by 7 equals a basic number of weeks value of 9.29. The method according to the presently disclosed subject matter further comprises the step of adding a factor of two (weeks) to the first or basic number of weeks value thereby yielding a second number of weeks value or an adjusted gestational maturity value in number of weeks.

In the case of the gestational maturity descriptor example, “kidney” with word value (20) of 65, the adjusted gestational maturity value or code2GOD adjustment value in number of weeks is 9.29+2=11.29. The adjusted gestational maturity value is then compared to secular gestational maturity data associated with the first gestational maturity descriptor for providing a generally minimized gestational difference value, which minimized gestational difference value may be used by the decoder to evidence intelligent design of human life by the author of the original Hebrew bible letter string.

Referencing FIG. 13, the reader will there consider various gestational maturity descriptors for each of the primary gestational organs. For example, the English gestational maturity descriptor, “stomach” or in Hebrew, “beten” or “ [symbol] ” is associated with a word value (20) of 61. The code2GOD adjustment value is 10.7 (=61÷7+2) roughly corresponding to a period of 11 weeks with a minimized gestational difference value of 3, which is the established gestational period of time for full stomach maturity when the intestine leaves the umbilical cord and returns to the abdomen and the rectum and the anus form.

The English gestational maturity descriptor, “brain” or in Hebrew, “moach” or “ [symbol] ” is associated with a word value (20) of 48. The code2GOD adjustment value is 8.8 (=48÷7+2) roughly corresponding to a period of 9 weeks with a minimized gestational difference value of 0.2, which is the established gestational period of time for full brain maturity. The brain stem and cerebellum connect the brain to the spinal cord and control the body's breathing, heart rate, blood pressure, balance, and reflexes. The limbic system sits on top of the brain stem and governs many different functions including emotion, thirst, hunger, memory, learning, and the body's daily rhythms. The cerebral cortex comprises a left and right hemisphere, and sits on top of the limbic system, and contains: an occipital lobe—for vision; a temporal lobe—for hearing, language and social interaction; a frontal lobe—for memory, self-regulation, planning and problem solving; and a parietal lobe—for bodily sensations like pain, pressure, heat and cold.

The English gestational maturity descriptor, “liver” or in Hebrew, “kaved” or “ [symbol] ” is associated with a word value (20) of 26. The code2GOD adjustment value is 5.7 (=26÷7+2) roughly corresponding to a period of 6 weeks with a minimized gestational difference value of 0.3, which is the established gestational period of time for full liver maturity. During hepatic hematopoiesis the gestational liver is colonized by undifferentiated hematopoietic stem cells (HSCs) that gradually differentiate and once mature, enter the circulatory system through the hepatic sinusoids. This starts in the fetal liver during the sixth week of gestation according to established medical information.

The English gestational maturity descriptor, “blood” or in Hebrew, “dam” or “ [symbol] ” is associated with a word value (20) of 44. The code2GOD adjustment value is 8.3 (=44÷7+2) roughly corresponding to a period of 8 weeks with a minimized gestational difference value of 0.3, which is the established gestational period of time when the heart pumps blood and can be heard. The English gestational maturity descriptor, “heart” or in Hebrew, “lev” or “ [symbol] ” is associated with a word value (20) of 32. The code2GOD adjustment value is 6.6 (=32÷7+2) roughly corresponding to a period of 7 weeks with a minimized gestational difference value of 0.4, which is the established gestational period of time when the 4-chamber fetal heart is finally formed.

The singular English gestational maturity descriptor, “eye” or in Hebrew, “ein” or “ [symbol] ” is associated with a word value (20) of 130. The code2GOD adjustment value is 20.5 (=130÷7+2) roughly corresponding to a period of 20 weeks with a minimized gestational difference value of 0.5, which is the established gestational period of time when the eyes are finally formed. The plural English gestational maturity descriptor, “eyes” or in Hebrew, “einayim” or “ [symbol] ” is associated with a word value (20) of 190. The code2GOD adjustment value is 29 (=190÷7+2) corresponding to a period of 29 weeks with a minimized gestational difference value of 0.0, which is the established gestational period of time when fetal eyes are able to open.

The English gestational maturity descriptor, “taste” or in Hebrew, “ta-am” or “ [symbol] ” is associated with a word value (20) of 119. The code2GOD adjustment value is 19 (=119÷7+2) roughly corresponding to a period of 20 weeks with a minimized gestational difference value of 1.0, which is the established gestational period of time when a developing fetus can taste nutritional matter as consumed by the mother and present in the amniotic fluid and taste bud signals to the brain are established. The English gestational maturity descriptor, “mouth” or in Hebrew, “pe” or “ [symbol] ” is associated with a word value (20) of 85. The code2GOD adjustment value is 14.1 (=85÷7+2) roughly corresponding to a period of 14 weeks with a minimized gestational difference value of 0.1, which is the established gestational period of time when the mouth of a developing fetus latches, sucks and exhibits chewing movements.

The plural English gestational maturity descriptor, “ears” or in Hebrew, “oznaim” or “ [symbol] ” is associated with a word value (20) of 124. The code2GOD adjustment value is 19.7 (=124÷7+2) roughly corresponding to a period of 20 weeks with a minimized gestational difference value of 0.3, which is the established gestational period of time when the fetal ears are finally formed and the fetus can perceive auditory information. The plural English gestational maturity descriptor, “hands” or in Hebrew, “yadaim” or “ [symbol] ” is associated with a word value (20) of 74. The code2GOD adjustment value is 12.6 (=74÷7+2) roughly corresponding to a period of 13 weeks with a minimized gestational difference value of 0.4 weeks, which is the established gestational period of time when the fetal hands have developed 27 bones connected by ligaments which are structurally complete.

The singular English gestational maturity descriptor, “bone” or in Hebrew, “etzem” or “ [symbol] ” is associated with a word value (20) of 200. The code2GOD adjustment value is 30.6 (=124÷7+2) roughly corresponding to a period of 30 weeks with a minimized gestational difference value of 0.4, which is the established gestational period of time when 200 bones and 6 auditory ossicles are finally formed. The English gestational maturity descriptor, “nose” or in Hebrew, “af” or “ [symbol] ” is associated with a word value (20) of 81. The code2GOD adjustment value is 13.6 (=81÷7+2) roughly corresponding to a period of 11-14 weeks with a minimized gestational difference value range of 0.4 to 2.6 weeks, which is the established gestational period of time when the nasal bone is finally formed.

The singular English gestational maturity descriptor, “kidney” or in Hebrew, “kilia” or “ [symbol] ” is associated with a word value (20) of 65 as noted above. The code2GOD adjustment value is 11.2 (=65÷7+2) roughly corresponding to a period of 11-12 weeks with a minimized gestational difference value of 0.2 to 0.8 weeks, which is the established gestational period of time when urine formation begins toward the end of the first trimester continues to gestationally develop. The singular English gestational maturity descriptor, “lung” or in Hebrew, “rei-a” or “ [symbol] ” is associated with a word value (20) of 216. The code2GOD adjustment value is 32.8 (=216÷7+2) roughly corresponding to a period of 32 weeks with a minimized gestational difference value of 0.2 weeks, which is the established gestational period of time when Stage 5 of lung development occurs. The English gestational maturity descriptor or expression, “digestive system” or in Hebrew, “ [symbol] ” is associated with a word value (20) of 136. The code2GOD adjustment value is 21.4 (=136÷7+2) roughly corresponding to a period of 23 weeks with a minimized gestational difference value of 1.6 weeks, which is the established gestational period of time when peristalsis or the involuntary constriction and relaxation of the muscles of the intestine or another canal, creating wave-like movements that push the contents of the canal forward.

The English gestational maturity descriptor, “fetus” or in Hebrew, “ubar” or “ [symbol] ” is associated with a word value (20) of 278. The code2GOD adjustment value is 41.7 (=278÷7+2) roughly corresponding to a period of 42 weeks with a minimized gestational difference value of 0.3 weeks, which is the established gestational period of time when gestation is complete. The English gestational maturity descriptor, “gender” or “sex” or in Hebrew, “meen” or “ [symbol] ” is associated with a word value (20) of 100. The code2GOD adjustment value is 16.3 (=100÷7+2) roughly corresponding to a period of 16 weeks with a minimized gestational difference value of 0.3 weeks, which is the established gestational period of time when gender may be reliably determined.

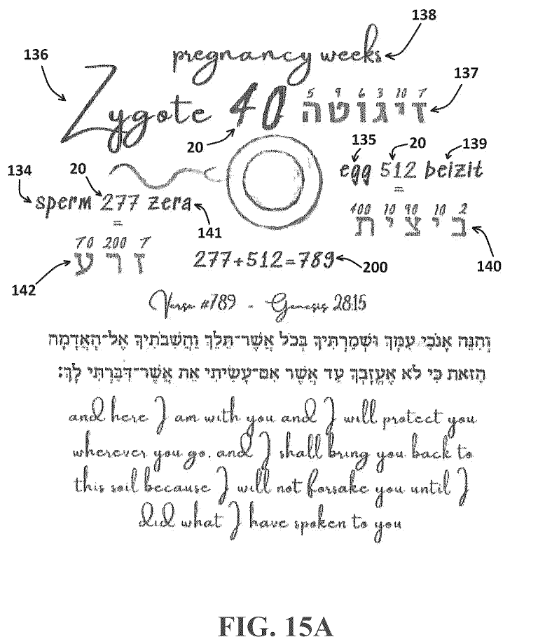

FIG. 14 presents a visual embodiment of the SPOG analytical system applied to human embryological development. As illustrated in FIG. 14, encoded linguistic-numeric values derived from Hebrew biological terms are correlated with fixed human developmental timelines corresponding to functional readiness of multiple organs, including organ-specific functionality milestones. The figure integrates: Hebrew words for organs and life-processes, each annotated with its code2GOD numeric value as defined in U.S. Pat. No. 12,400,087; an anatomical illustration of a developing human embryo; numeric indicators corresponding to the exact day from conception on which each organ becomes functionally mature; a central demonstration of the Hebrew term [symbol] (pregnancy) yielding a code2GOD value of 271, equal to the gestation period of 271 days; and correlated developmental milestones including heart (32), kidney (65), brain (48), stomach (61), ears (128), hands (14), and bone (200). This figure visually consolidates the biological relationships derived through the SPOG method, allowing a viewer to assess encoded linguistic values and their correspondence to empirically verified developmental constants in a single integrated display.

Referencing FIG. 14, the reader will there consider the concept of pregnancy as primarily denoted by the English word “pregnancy” (113) or in Hebrew, “heraion” (114) or “ [symbol] ” (115). The Hebrew word, “ [symbol] ” (115) has a word value (20) of 271 corresponding to the average number of days of gestation, conception to birth, based on 10,000 births. The Hebrew word, “ [symbol] ” (116), derived from the Hebrew word, “ [symbol] ” (115) has a word value (20) of 66 based on Code Nos. 1 and 2 of the basic foundational code2GOD methodology according to the presently disclosed subject matter, and carries several meanings. A first English term is “dove” (117) or in Hebrew, “yon”, is a symbolic gesture representing umbilical cord cutting and detaching the baby to his/her own life (133). An alternative meaning in English is the term, “ion” with similar representation in Hebrew “ [symbol] ” (118) and means unbalanced, charged, progressing (131). The English descriptors, “new” or “beautiful” (127) or in Hebrew, “noi” (128) or “ [symbol] ” (119) comprises the Hebrew word, “ [symbol] ” (120) read in reverse and is an expression of surprise (129).

The first two letters of the English, “pregnancy” (113) in GOD's Biblical Hebrew “heraion” (114) or “ [symbol] ” (115) are “ [symbol] ” (121) which means a mountain (132). Based on the basic code2GOD methodology according to the presently disclosed subject matter, the Hebrew, “ [symbol] ” (121) has a word value (20) of 205 which is also the sentence value for “LORD GOD” or “holy GOD the LORD protects” (130) and in Hebrew, “ [symbol] ” (112). Based on code2GOD's factor of the Chosen One, an interpretation derived from the word/sentence values yields a “mountain of imbalance in progress” (123) as an equivalent interpretation of “pregnancy” (113). The English descriptor, “parents” (124) or in Hebrew, “horim” (125) or “ [symbol] ” (126) has a word value (20) of 261 which is a word value (20) 10 less than the word value (20) for “pregnancy” (11) or in Hebrew, “heraion” (114) or “ [symbol] ” (115). The English descriptor, “fetus” or in Hebrew, “ubar” or “ [symbol] ” has a word value (20) of 278 which word value (20) is 7 greater than the word value (20) of “pregnancy” (113) or in Hebrew, “heraion” (114) or “ [symbol] ” (115). It will thus be seen that pregnancy may be tied to other descriptors such as parents or fetus by way of word value (20) similarities and can be used to derive secondary or code2GOD meaning from the original Hebrew bible letter string as evidence of an intelligent designer or author of the original Hebrew bible letter string.

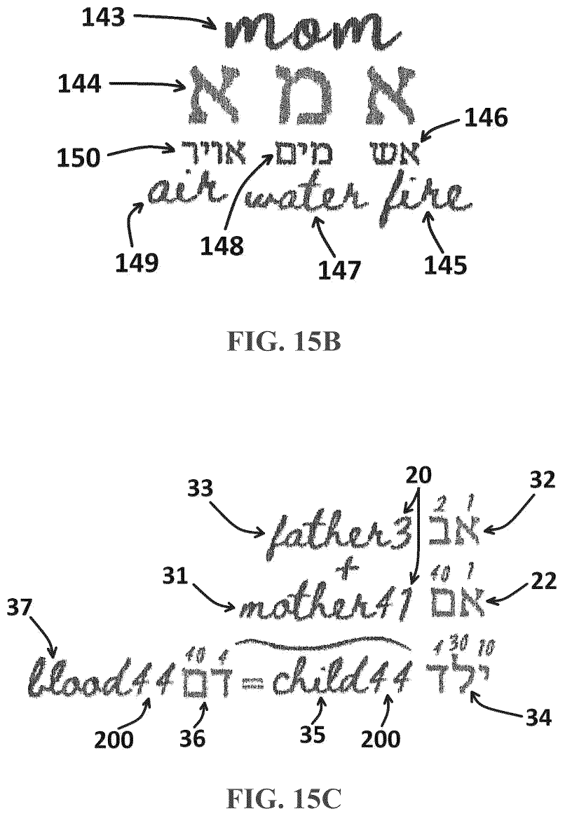

Referring to FIG. 3, a medical calibration system is shown in which clinical developmental observations based on estimated conception timing are aligned against fixed organ-functionality benchmarks to quantify estimation error and generate calibrated developmental assessments. Considering FIG. 15A, the reader will there consider the moment the sperm (134) penetrates the egg (135) resulting in a fertilized egg or “zygote” (136) or in Hebrew, “zigota” or “ [symbol] ” (137) having a word value (20) of 40, which numerical word value (20) is equivalent to the average length of a “pregnancy” (113) (conception to birth) as measured in weeks (138). The English word, “sperm” (134) or in Hebrew, “zera” (141) or “ [symbol] ” (142) has a word value (20) of 277. The English word, “egg” (135) or in Hebrew, “beizit” (139) or “ [symbol] ” (140) has a word value (20) of 512. Adding the word value (20) of “sperm” (134) or 277 to the word value (20) of “egg” (135) or 512 yields a total combination word value (200) of 789. Using the code2GOD methodology according to the presently disclosed subject matter, it will be noted that verse 789 in Genesis 28:15 reads, as follows in the original Hebrew bible juxtaposed against the English translation: [symbol] [symbol] [symbol] [symbol]
and here I am with you and I will protect you wherever you go, and I shall bring you back to this soil because I will not forsake you until I did what I have spoken to you

As illustrated in FIG. 4, a method for determining fetal life status is implemented by selecting relevant organs, retrieving fixed functionality milestones, evaluating developmental observations, aggregating organ-level results, and outputting a life-status determination based on functional readiness. Considering FIG. 15B, it will there be seen that the English word, “mom” (143) is “eema” or “ [symbol] ” (144) in Hebrew. The Hebrew letter, “ [symbol] ” can stand for the English word, “fire” (145) or in Hebrew, “esh” or “ [symbol] ” (146) having a word value (20) of 301. The Hebrew letter, “ [symbol] ” can stand for the English word, “water” (147) or in Hebrew, “ma-im” or “ [symbol] ” (148) having a word value (20) of 90. The third Hebrew letter, “ [symbol] ” can stand for the English word, “air” (149) or in Hebrew, “avir” or “ [symbol] ” (150) having a word value (20) of 217. The combination word value (200) for the English terms, “air” (149), “water” (147), and “fire” (145) is 608 based on the code2GOD methodology according to the presently disclosed subject matter.

Another numerically based messaging possibility for the English word, “mom” (143) or in Hebrew, “eema” or “ [symbol] ” (144) is illustrated by the following example. The Hebrew letter, “ [symbol] ” can stand for the English word, “fire” (145) or in Hebrew, “esh” or “ [symbol] ” (146) having a word value (20) of 301. The Hebrew letter, “ [symbol] ” can stand for the English word, “water” (147) or in Hebrew, “ma-im” or “ [symbol] ” (148) having a word value (20) of 90. The third Hebrew letter, “ [symbol] ” can stand for the English word, “land” (46) or in Hebrew, “adama” (45) or “ [symbol] ” (44) having a word value (20) of 50. The combination word value (200) for the English terms, “air” (149), “water” (147), and “land” (46) is 441 based on the code2GOD methodology according to the presently disclosed subject matter. The numerical value 441 is equivalent to the English word, “truth” (65) or in Hebrew, “emet” (64) or “ [symbol] ” (63).

Another numerically based messaging possibility for the English word, “mom” (143) or in Hebrew, “eema” or “ [symbol] ” (144) is illustrated by the following example. The Hebrew letter, “ [symbol] ” can stand for the English word, “land” (46) or in Hebrew, “adama” (45) or “ [symbol] ” (44) having a word value of 50. The Hebrew letter, “ [symbol] ” can stand for the English word, “water” (147) or in Hebrew, “ma-im” or “ [symbol] ” (148) having a word value (20) of 90. The third Hebrew letter, “ [symbol] ” can stand for the English word, “air” (149) or in Hebrew, “avir” or “ [symbol] ” (150) having a word value (20) of 217. In this example, the combination word value (200) for the English terms, “air” (149), “water” (147), and “land” (46) is 357 based on the code2GOD methodology according to the presently disclosed subject matter.

Comparatively referencing FIGS. 3C and 15C, the reader will recall the word value (20) for the Hebrew term, “ [symbol] ” (22) (or in English, “mother” (31)) equals 41 and that the word value (20) for the Hebrew term, “ [symbol] ” (32) (or in English, “father” (33)) equals 3. Adding the word values (20) of “ [symbol] ” or 41 and “ [symbol] ” or 3 equals a combination word value (200) of 44, which combination word value (200) corresponds to either the Hebrew term, “ [symbol] ” (34) or in English, “child” (35), or the Hebrew term, “ [symbol] ” (36) or in English, “blood” (37). When interpreted by a chosen interpreter, the resulting secondary or code2GOD meaning may translate into a message that a “mother” (31) and a “father” (33) together yield a “child” (35) or alternatively, “blood” line (37).

Noting that various organs of the human body may either develop in multiples or pairs, the code2GOD system/method according to the presently disclosed subject matter further contemplates that select word values (20) for the gestational maturity descriptors can be multiplied by an organ number factor for yielding an organ product value. For example, the word value (20) of 65 for “kidney” or “kilia” or “ [symbol] ”, may be multiplied by an organ factor number being equal to 2 (i.e. the number of anatomical kidneys) associated with the gestational maturity descriptor. In this case, the organ product value equals 130 and this numerical product value provides additional numerical information for supporting secondary messaging from the original Hebrew bible letter string.

Further, the gestational maturity descriptor(s) may be pluralized to form a pluralized gestational maturity descriptor as in the case of a pair of or two kidneys, or 10 fingers and 10 toes. The pluralized gestational maturity descriptors each have a pluralized word value different than the select word value of the basic gestational maturity descriptor (the singular term, “finger” is different than the plural “fingers”) for providing additional numerical information and supporting secondary messaging from the original Hebrew bible letter string.

FIG. 16 depicts the SPOG analytical system applied to Hebrew terms associated with spiritual concepts and origin-of-life processes. The figure includes: the code2GOD numeric breakdown of Hebrew terms such as “mother,” “father,” “blood,” “zygote,” “sperm,” and “egg,” each rendered with its corresponding numeric value; a depiction of the fertilization process and formation of the zygote, annotated with the relevant code2GOD totals; a representation of combined numeric values, such as sperm (277) plus egg (512) yielding 789; and scriptural references illustrating how encoded linguistic values map to textual structures. This figure provides a visualization of the SPOG system as it applies to conceptual, spiritual, and generative terms, demonstrating how non-biological Hebrew words also contain structured numeric values that correlate with definable scientific and textual parameters.

FIG. 17 shows a large-format framed artwork embodying the complete SPOG system in a single physical presentation. Referring to FIG. 17, a scientific visualization framework is illustrated that integrates biological development data, encoded linguistic-numeric values, and fixed developmental timelines to visually demonstrate invariant developmental consistency across organs and stages of human development. The figure includes: the full composite rendering of encoded pregnancy (271), organ-development timelines, spiritual encoding tables, and the visual depiction of the Hebrew Bible; integration of FIGS. 14 and 16 into a unified scientific-visual panel; a large physical frame measuring approximately five feet in width, designed for educational, scientific, demonstrative, and museum display; and the placement of numeric, textual, anatomical, artistic, and linguistic components arranged to convey the SPOG analysis as a cohesive scientific visualization. This artwork constitutes a tangible embodiment of the SPOG analytical system and serves as a comprehensive visualization tool by presenting all primary SPOG findings—linguistic, biological, and conceptual—in a single integrated display medium.

While the foregoing descriptions contain certain specificity, this specificity should not be construed as limitations on the scope of the presently disclosed subject matter, but rather as an exemplification thereof. The foregoing specifications are contemplated to essentially provide a system and method, optionally implemented by at least one computer and a non-transitory software-based application implementable by at least one computer for deriving secondary or code2GOD meaning from a select letter string as exemplified by the original Hebrew biblical string consisting of 1,197,000 Hebrew letters devoid of spaces and punctuation. When considered methodologically, the method may be said to essentially comprise a series of steps. When considered systemically, the system according to the presently disclosed subject matter may be said to essentially comprise at least one computer and a non-transitory computer-implementable application for implementing the essential methodology.

The method for deriving secondary or code2GOD meaning from a select letter string essentially comprises the step of firstly identifying a primary sequence of Hebrew letters, each letter within the primary sequence of Hebrew letters shown Column 10 are associated with a select numerical letter value as shown in Column 11 of FIG. 1. The Hebrew letters within the primary sequence of letters as in Column 10 are arranged to form a select letter string as exemplified by the by the original Hebrew biblical string consisting of 1,197,000 Hebrew letters devoid of spaces and punctuation. The select letter string has a string value equal to the sum of the select numerical letter values for each of the Hebrew letters within the select Hebrew letter string.

The code2GOD system/method according to the presently disclosed subject matter functions to provide evidence of the intelligent design of human life as determined by gestational maturity information as authored by an intelligent designer and set forth within the original Hebrew bible letter string. The code2GOD system/method further comprises the step of identifying at least one gestational maturity descriptor or first word within the select letter string. Word values of select words may be correlated or loosely associated with other secondary or peripheral word values by way of the same or similar word values. The primary or face value meaning of firstly considered gestational maturity descriptors or words can then be interpreted in the context of the secondary or peripheral word values for providing at least one secondary meaning of select gestational maturity descriptors or words as compared to the primary or face value meaning(s) for bolstering an understanding of the primary meaning(s).

Once the gestational maturity descriptor(s) are identified within the original Hebrew bible letter string, and a word value is determined for each gestational maturity descriptor, each word value can be divided by a factor of seven to yield a first number of weeks value. A factor of two (weeks) is then added to the first number of weeks value for yielding a second number of weeks value or an adjusted gestational maturity value in number of weeks. The second number of weeks value or adjusted gestational maturity value is then compared to secular gestational maturity data aligned with the gestational maturity descriptor(s) for providing a minimized gestational maturity difference value, the negligibility of which evidences intelligent design of human life by an author or intelligent designer of the original Hebrew bible letter string.

Noting that various organs of the human body may either develop in multiples or pairs, the code2GOD system/method according to the presently disclosed subject matter further contemplates that select word values for the gestational maturity descriptors can be multiplied by an organ number factor for yielding an organ product value, the organ factor number being equal to the number of organs associated with the gestational maturity descriptor(s), and the organ product value for providing additional numerical information and supporting secondary messaging from the original Hebrew bible letter string. Further, the gestational maturity descriptor(s) may be pluralized to form a pluralized gestational maturity descriptor. The pluralized gestational maturity descriptors each have a pluralized word value different than the select word value for providing additional numerical information and supporting secondary messaging from the original Hebrew bible letter string.

Anagrams of the gestational maturity descriptors will always have the same word value of respective gestational maturity descriptors or words. In other words, if the letters of the at least one first word are re-arranged to form at least one anagram word, the at least one anagram word comprises the select word value or at least two secondary sub word values if the first select word is re-arranged and broken into a series of anagram words derived from the first select word. The word values for the first select word and its anagram(s) may be interpreted to further bolster an understanding of the gestational maturity descriptor by way of providing additional numerical information and attendant secondary messaging as derived from the original Hebrew bible letter string.

The code2GOD method according to the presently disclosed subject matter may further comprise the step of coupling the first select word value to at least one second word value to form a sequence or series of word values, which respectively can provide tertiary meaning or additional secondary or code2GOD meaning as interpreted within the context of multiple word values. The first word value and the at least one second word values may be correlated or otherwise arithmetically operated by way of a select arithmetic operator as selected from the group consisting of addition, subtraction, division and multiplication functions. The select arithmetic operator essentially outputs an arithmetically-operated word value, which arithmetically-operated word value yields or provides at least one arithmetically-operated word meaning for providing additional secondary or code2GOD meaning.

The relative or absolute position of select letters within the primary sequence of letters can be selected to form either a relative position word value or an absolute position word value. In this regard, the reader will recall that the Hebrew word or “truth” comprises the first Hebrew letter in Column 10, the last Hebrew letter in Column 10, and the 14^{th} or middle Hebrew letter in Column 10. An interpretation of the positions of these letters is that truth is not definite and can be found within the entire spectrum or listing of values associated with the listed or primary sequence of Hebrew letters. Similarly, word values may be characterized by select traits as selected from the group consisting of a mystery word value, a dominant word value, a meaningful word value, and a repetitive word value. An example of this is the value 37 which value is consistent with a normalized body temperature in Celsius degrees. Additional secondary or code2GOD meaning can be derived from these select traits.

The code2GOD system/method according to the presently disclosed subject matter may further focus upon a select string of words as selected from a select portion of an opening stanza of the select letter string. A first letter from each word of the select string of words are associated with a first letter value, and these first letter values may be totaled or summed and further operated by dividing the total number of letters for yielding a meaningful average numerical value, which meaningful average numerical value is substantially equal to 3.14 or π. This average numerical value being substantially equal to the 3.14 or π can provide additional secondary or code2GOD meaning for the select string of words as selected from a select portion of an opening stanza of the select letter string as exemplified by the original Hebrew biblical string consisting of 1,197,000 Hebrew letters devoid of spaces and punctuation. Select word values may also be logarithmically operated to the base of Euler's number e for providing a natural logarithm for select word values for deriving additional secondary or code2GOD meaning.

The code2GOD system/method according to the presently disclosed subject matter may further involve the summation of select numerical letter values of each letter in a select series of letters to provide a single reduced numerical value, the single reduced numerical value being either (a) between a range of values, the range of values being from 1 to 9; or (b) a select unique numerical value, the select unique numerical value being selected from the group consisting of 11, 22, 33, and 44. The single reduced numerical value can be used to derive additional secondary or code2GOD meaning for words having the same or related reduced numerical values as so reduced. It will be recalled that the reduced numerical value of 10 may represent perfection or may further reduce to a value of 1.

The code2GOD system/method according to the presently disclosed subject matter may further focus upon a series of factors that are utilized to operate upon a select letter sub-string within the select letter string. These series of factors may comprise or consist of acronym-forming letters; select intervals of letters; and directional movement within the select letter string, the series of factors for providing additional secondary or code2GOD meaning (exemplified by DNA information). It is contemplated that a computer and a software-based application may well provide the most efficient means for deriving secondary or code2GOD meaning from portions of the original Hebrew Biblical string very much akin to the manner in which genetic traits within human DNA are located.

The code2GOD system/method according to the presently disclosed subject matter may further focus upon the use of geometrical data to operate upon the select word value for providing additional secondary or code2GOD meaning. Geometrical data associated with the Star of David, for example, can be utilized to confirm the importance of the numerical value 37. In this regard, it will be recalled the Star of David Theorem is a mathematical result based on arithmetic properties of binomial coefficients. Comparatively referencing FIGS. 12A and 12B it will there be seen that the numbers 37 and 73 are inherently represented by the Star of David theorem, and can thus be harnessed to convey or derive secondary meaning from the original Hebrew bible letter string.

The code2GOD system/method according to the presently disclosed subject matter may further comprise the step of utilizing a select count to operate upon the select word value. The select count factor may be selected from the group consisting of a number of letters or letter number within a chosen letter string; a number of letters or letter number within related word strings; and a number of letters or letter number within a sentence. In this regard, it will be recalled the number of singular word repetitions in the original Hebrew Bible string can provide meaningful word values. Further, matching the first and last letters of a Hebrew word with the first and last letters of a verse from the original Hebrew bible string provides information for deriving additional secondary or code2GOD meaning. Further, using prime or Fibonacci numbers to explain correlations between Hebrew words or verses provides information for deriving additional secondary or code2GOD meaning.

The code2GOD system/method according to the presently disclosed subject matter may focus upon the fact that each Hebrew letter within the primary sequence of Hebrew letters comprises a unique size and shape. The unique size and shape of each letter provides factor data, which factor data may be selected from the group consisting of letter termini data (i.e. number of end points); directional termini data (i.e. the number of end points in each of the four primary directions); and relative height-width data (the relative size of comparable Hebrew letters). The factor data can provide additional secondary or code2GOD meaning.

A number of utilitarian aspects deriving from the presently disclosed subject matter are discussed in more detail hereinafter. A first utilitarian feature according to the presently disclosed subject matter relates to SPOG as Verification of the patented code2GOD System as a Valid Scientific Method for Understanding the Divine. SPOG employs the patented code2GOD Scientific System Decoder (U.S. Pat. No. 12,400,087) to validate that the Hebrew letter-to-number assignments yield reproducible, measurable correlations with biological and physical constants. This confirmation establishes code2GOD as a verifiable scientific instrument for exploring encoded knowledge within the Hebrew language. Because only a Creator could have embedded accurate biological constants in language thousands of years ago, SPOG provides confirmation that code2GOD is a valid scientific path to understand the Divine Mind behind creation.

A second utilitarian feature according to the presently disclosed subject matter relates to SPOG as Defining God as the Designer of Life. By demonstrating exact numeric correspondences between Hebrew words for human organs and the number of days it takes each organ to reach functionality (e.g., [symbol] “heart”=32 days), SPOG produces measurable evidence of deliberate design in human biology. The precision of these values proves that God designed the structure and timeline of human development, making Him definable scientifically as the Designer of life.

A third utilitarian feature according to the presently disclosed subject matter relates to SPOG as Demonstrating that God Preserves His Design Beyond Evolutionary Change. SPOG shows that biological constants—such as the 271-day gestation period—have remained unchanged across human history. This invariance contradicts expected evolutionary drift and demonstrates the continuing stability of the original design. Because these parameters have never changed, God is seen as protecting and maintaining His design, preserving life's original blueprint despite natural evolutionary forces.

A fourth utilitarian feature according to the presently disclosed subject matter relates to The Hebrew Language: Created and Encoded by God as a Communication System with Humanity. SPOG reveals that Hebrew (Ivrit, from avar, “to transfer”) functions as a divinely engineered medium for transferring information. Its dual numeric structure allows the extraction of scientific data, confirming intentional encoding by an intelligent author. The structure of Hebrew itself is the message—God's original language for communication with humanity, embedding knowledge for later discovery.

A fifth utilitarian feature according to the presently disclosed subject matter relates to The Hebrew Language: Encoded with Scientific Information Beyond Modern Science. SPOG analysis demonstrates that many Hebrew words contain numerical information corresponding to scientific constants unknown at the time of the language's formation. These include biological durations, cosmic ratios, and physical relationships. This proves that God encoded scientific knowledge within the Hebrew language thousands of years before its human discovery.

A sixth utilitarian feature according to the presently disclosed subject matter relates to The Hebrew Bible: The Foundational Text Containing Encoded Scientific Data. Application of the SPOG method to the original Hebrew Bible reveals that its text systematically embeds numeric data aligning with biological and physical realities. These correlations demonstrate structured encoding rather than random occurrence. The Hebrew Bible thus serves not only as a moral and spiritual guide but also as a scientific record authored by God, containing encoded truths that surpass today's science.

A seventh utilitarian feature according to the presently disclosed subject matter relates to SPOG as Scientific Proof that Humanity Did Not Evolve from an Amoeba. Because SPOG confirms that all human organ-development timelines and reproductive constants are fixed and precisely encoded, it disproves the possibility of random Darwinian evolution from lower organisms. Humanity was created according to a divine plan, not as the result of spontaneous biological evolution.

An eighth utilitarian feature according to the presently disclosed subject matter relates to SPOG as a Medical Utility for Determining Organ Development Timelines. SPOG provides a reproducible data table mapping Hebrew organ names to their code2GOD values, corresponding exactly to the number of days between conception and organ functionality. This enables medical researchers to calibrate and verify developmental stages with greater precision than current empirical methods. Where medical science can only estimate, the Designer of life knows precisely when each organ becomes functional. SPOG aligns medical understanding with that divine precision.

A ninth utilitarian feature according to the presently disclosed subject matter relates to SPOG as a Utility for Understanding Divine Symmetry in Dual Organs. SPOG reveals mathematical and linguistic symmetry in Hebrew terms for paired organs—such as kidneys, lungs, and eyes—mirroring their anatomical and functional duality. This symmetry demonstrates God's intentional balance and redundancy in human design, offering new insights into physiological harmony.

A tenth utilitarian feature according to the presently disclosed subject matter relates to SPOG as Proof that God is Superior to Nature and Evolution. By showing that gestation time and organ development have been shielded from evolutionary variation, SPOG demonstrates that biological processes operate under fixed design parameters rather than random natural selection. This proves that God's design governs and surpasses nature, maintaining constancy where evolution predicts change.

An eleventh utilitarian feature according to the presently disclosed subject matter relates to SPOG as a Medical Calibration Utility for Clinical Research. SPOG provides a divine reference model for conception and organ functionality. Modern medicine, unable to determine exact conception timing, relies on rough estimates (last menstrual period, average cycle), leading to deviations of up to three weeks. SPOG supplies precise, encoded timing benchmarks (e.g., kidneys=65 days, heart=32 days, brain=48 days), allowing calibration of clinical studies against the Creator's design. Where current science is approximate, God's encoded system provides exact truth—offering doctors and researchers a fixed standard for understanding human development.

A twelfth utilitarian feature according to the presently disclosed subject matter relates to SPOG as a Defining Tool When an Organ Is Functional According to the Creator. SPOG identifies the divine threshold of organ functionality based on encoded timing, such as the lungs reaching their fifth stage of maturity months after birth. This offers a new metric for medical and developmental research. It defines when God considers an organ to be “alive” or fully functional, providing a divine reference point for biological life stages.

A thirteenth utilitarian feature according to the presently disclosed subject matter relates to SPOG as a Utility for Pro-Life and Ethical Policy Determination. SPOG provides objective, encoded data on when fetal organs become functional, creating a measurable framework for determining the onset of life in scientific, ethical, and governmental contexts. It gives pro-life and medical communities a scientific basis derived from divine design to establish when life begins, merging empirical data with spiritual truth.

A fourteenth utilitarian feature according to the presently disclosed subject matter relates to SPOG as a Utility for Data Authentication and Integrity Verification of Biblical Texts. Because the Hebrew code2GOD values create consistent numeric relationships, SPOG can be used as a checksum system to detect textual errors or later edits in ancient manuscripts. By confirming the internal numeric harmony, it authenticates which versions of a verse preserve the original encoded design. This function protects the divine message from corruption, showing that God embedded a self-verifying mechanism inside His language.

A fifteenth utilitarian feature according to the presently disclosed subject matter relates to SPOG as a Utility for Artificial-Intelligence Modeling of Divine Design. The mathematical regularities uncovered by SPOG can train AI or machine-learning models to recognize patterns of “intentional design” across biological or linguistic data sets. It transforms the qualitative idea of “divine order” into measurable, learnable parameters. By digitizing the signatures of divine design, SPOG allows modern technology to study and replicate the patterns of God's creation in a controlled scientific way.

A sixteenth utilitarian feature according to the presently disclosed subject matter relates to SPOG as a Utility for Universal Information-Encoding Standards. The code2GOD method shows that a finite symbolic system can carry unlimited layers of meaning—literal, numerical, and functional. This principle can guide the design of next-generation information systems or communication codes that mirror the efficiency of divine language. Humanity can model its own communication systems on the perfect informational architecture created by God, using SPOG as the template.

A seventeenth utilitarian feature according to the presently disclosed subject matter relates to SPOG as a Utility for Integrating the Spiritual and Scientific Realms. SPOG introduces a structured methodology that converts spiritually described concepts into quantifiable, testable data. By using the code2GOD Scientific System Decoder to assign numeric values to words describing spiritual realities—such as soul ( [symbol] ), spirit ( [symbol] ), or creation ( [symbol] )—and comparing those values with physical and biological constants, the system provides a unified data layer between metaphysical terminology and scientific observation. This enables measurable dialogue between theology, philosophy, and empirical science. Through this process, SPOG functions as a bridge between the spiritual and scientific worlds. It demonstrates that spiritual truths are not abstract beliefs but encodable, discoverable information within the structure of creation. Thus, SPOG allows humanity to perceive how God's spiritual design is mathematically present in the physical universe—reuniting faith and science under one coherent system.

A first aspect according to the presently disclosed subject matter is a Method for Validating the code2GOD System as a Scientific Analytical Tool. 1. A method for producing reproducible numeric correlations between Hebrew lexical data and biological constants, comprises: (a) accessing a database containing the twenty-two Hebrew letters with the numeric assignments defined in U.S. Pat. No. 12,400,087 (“code2GOD Scientific System Decoder”); (b) selecting a Hebrew word that designates a biological organ or process; (c) calculating the total code2GOD value of the word by summing the assigned numeric values of its constituent letters; and (d) comparing the resulting total with a verified biological measurement corresponding to the named organ or process.

In some applications, the Hebrew word: [symbol] (kilya, “kidney”) produces a code2GOD value of 65 and the empirical constant represents 65 days after conception when renal filtration first becomes functional in the human embryo. In some applications, correspondence between the calculated value and the biological constant within a predetermined tolerance range constitutes validation that the code2GOD Scientific System Decoder yields measurable, non-random results. In some embodiments, when applied to additional terms such as [symbol] (herayon, “pregnancy”)=271 days or [symbol] (lev, “heart”)=32 days, the calculated values match the precise developmental timelines observed in human gestation—the point at which the kidneys begin filtration, the heart forms four chambers and begins coordinated contractions, and pregnancy reaches full term. Because these numeric correspondences are reproducible across multiple organs, they validate the code2GOD method as a genuine scientific tool for extracting biological information encoded in the Hebrew language.

A second aspect according to the presently disclosed subject matter is a Method for Demonstrating Exact Design in Human Biology. A method for demonstrating fixed, non-random relationships between Hebrew lexical data and human biological development, comprises: (a) applying the code2GOD Scientific System Decoder defined in U.S. Pat. No. 12,400,087 to Hebrew words that designate human organs and developmental processes; (b) calculating, for each selected Hebrew word, a total numeric value derived from the assigned numeric values of its constituent letters; (c) comparing each calculated total with the verified biological duration, measured in days from conception to the moment the corresponding organ becomes functionally mature; and (d) recording the set of word-duration correspondences for all examined organs.

In some applications, the Hebrew word [symbol] (herayon, “pregnancy”) yields a total of 271 and the biological constant is 271 days of gestation. In some applications, the Hebrew word [symbol] (lev, “heart”) yields 32 and the biological constant is 32 days from conception to the stage at which the heart has four chambers and performs its first coordinated beat. In some applications, the Hebrew word [symbol] (kilya, “kidney”) yields 65 and the biological constant is 65 days from conception to the onset of renal filtration. In some applications, verified matches are established for each major human organ, producing a complete table of exact numerical equivalence between linguistic values and biological functionality. When this method is applied to all organs named in the Hebrew lexicon, every calculated code2GOD value exactly equals the number of days from conception to the organ's moment of functional maturity. No statistical variation or random coincidence is observed. The author interprets this perfect correspondence across the entire human anatomy as evidence of an exact design encoded intentionally within the Hebrew language—a design that modern science can now measure but not originate. Therefore, according to the author, the SPOG system defines God, the Designer of life, as the only intelligence capable of such precise and universal encoding.

A third aspect according to the presently disclosed subject matter is a Method for Detecting Persistent Invariance in Human Biological Development. A method for identifying biological constants that remain unchanged across recorded history, comprises: (a) applying the code2GOD Scientific System Decoder of U.S. Pat. No. 12,400,087 to Hebrew lexical data describing human organs and developmental processes; (b) calculating, for each word, the total code2GOD value as the sum of the numeric assignments of its constituent letters; (c) compiling historical biological measurements for the corresponding organs from modern embryological studies and ancient medical records; (d) comparing each calculated value with the historical and contemporary measurements; and (e) identifying every case in which the measured constant remains identical to the encoded numeric value, thereby confirming persistence of the design parameter.

In some applications, the Hebrew word [symbol] (herayon, “pregnancy”) yields 271 and the empirical gestation period in all observed populations is 271 days. In some applications, each Hebrew organ name produces a numeric value identical to the number of days from conception to functional maturity for that organ, and said values remain constant in all available medical data. In some applications, a stability index is generated that quantifies the absence of evolutionary variance among the encoded biological constants. When the encoded values for every organ and life-process word are compared with present-day biological data, all values remain fixed and identical through time. Gestation has always been 271 days; the heart becomes functional on day 32; the kidneys filter on day 65, and so on. No drift, mutation, or environmental adaptation alters these numbers. The author interprets this perfect invariance as showing that the blueprint of human life has been preserved intact since its origin. In the author's view, such perpetual stability cannot arise from undirected evolution and therefore indicates the continuing preservation of the design by the original Designer of life.

A fourth aspect according to the presently disclosed subject matter is a Method for Detecting Structured Encoding within the Hebrew Language. A method for identifying non-random, information-bearing structure in the Hebrew language, comprises: (a) accessing a database containing the twenty-two Hebrew letters with the numeric assignments defined in U.S. Pat. No. 12,400,087 (code2GOD Scientific System Decoder); (b) selecting a corpus of Hebrew words drawn from classical or biblical texts; (c) calculating, for each word, a total code2GOD numeric value; (d) analyzing the distribution of the calculated values for statistically significant relationships to empirically verified natural constants or biological data; and (e) identifying patterns of correlation that exceed random probability thresholds.

In some applications, correlations include but are not limited to numerical matches between Hebrew words describing biological functions and the corresponding quantitative values of those functions in nature. In some applications, identified correlations are stored as an encoded-knowledge dataset for linguistic, scientific, and historical analysis. When the code2GOD analysis is applied to Hebrew words describing life and creation, the numeric totals consistently align with measurable facts in biology and physics. For example, words denoting organs, astronomical bodies, and natural phenomena each yield numbers that reflect the very quantities or durations associated with those entities. The author interprets these reproducible patterns as showing that the Hebrew language is a deliberately constructed communication system—a channel through which the Creator embedded scientific knowledge for humanity to discover. Under this interpretation, the language functions as a bridge between divine design and human understanding, confirming that its structure was authored intentionally rather than evolved naturally.

A fifth aspect according to the presently disclosed subject matter is a Method for Detecting Encoded Scientific Information in the Hebrew Language. A method for identifying quantitative correspondences between Hebrew lexical data and verified scientific constants, comprises: (a) retrieving the numeric assignments of the twenty-two Hebrew letters as defined in U.S. Pat. No. 12,400,087 (code2GOD Scientific System Decoder); (b) calculating, for each selected Hebrew word, a total numeric value representing the sum of its constituent letters; (c) comparing each calculated value with empirically established scientific constants selected from the group consisting of biological durations, astronomical periods, physical ratios, or mathematical constants; and (d) recording those Hebrew words whose calculated values equal or proportionally correspond to the verified constants.

In some applications, a Hebrew term describing a biological process yields a numeric value equal to the measured duration of that process in days. In some applications, a Hebrew term describing a physical or astronomical phenomenon yields a numeric value equal to the corresponding physical constant or orbital period when expressed in the same numeric scale. In some applications, a statistical report may be generated quantifying the probability that the detected correspondences occur by chance. Applying this method to a broad set of Hebrew words reveals that many contain numbers identical to modern scientific facts—values that were unknown when the language first appeared.

For instance, life-related terms produce the exact day counts of organ functionality, while words describing the heavens align with astronomical or physical constants. Because these matches are systematic and reproducible, the author interprets them as showing that scientific information was intentionally encoded in the Hebrew language. Under this interpretation, the language functions as a repository of knowledge far exceeding the scientific understanding of the era of its composition, demonstrating that the Author of the language possessed complete knowledge of nature and life.

A sixth aspect according to the presently disclosed subject matter is a Method for Detecting Encoded Scientific Data in the Hebrew Bible. A method for analyzing a Hebrew text corpus to identify embedded quantitative information, comprises: (a) accessing a digital or printed corpus of the Hebrew Bible; (b) applying the code2GOD Scientific System Decoder of U.S. Pat. No. 12,400,087 to calculate numeric values for each word or phrase within the corpus; (c) comparing the calculated values with verified scientific constants, measurements, or durations drawn from the fields of biology, astronomy, mathematics, and physics; (d) recording instances in which the calculated values equal or proportionally correspond to said verified constants; and (e) storing the identified correlations as an encoded-knowledge index for further linguistic and scientific study.

In some applications, the analyzed words include Hebrew terms describing biological processes whose numeric totals correspond exactly to the number of days required for those processes to occur. In some applications, statistical analysis demonstrates that the frequency of correspondence between textual values and scientific constants exceeds random probability thresholds. In some embodiments, a report may be listing the verified correspondences and their associated scriptural references. When this procedure is applied to the original Hebrew Bible, numerous words and verses yield numbers that match precise, modern scientific facts—such as the length of human gestation, organ-development milestones, and measurable physical ratios. These matches appear consistently across the text, not as isolated coincidences. The author interprets this pattern as showing that the Hebrew Bible contains deliberately encoded scientific information placed there through a structured linguistic system. Under this interpretation, the text operates simultaneously as a moral, spiritual, and scientific document, authored by an intelligence possessing complete understanding of life and the universe.

A seventh aspect according to the presently disclosed subject matter is a Method for Testing the Stability of Human Biological Constants Across Evolutionary Time. A method for determining whether specific human biological parameters have changed over evolutionary time, comprises: (a) applying the code2GOD Scientific System Decoder of U.S. Pat. No. 12,400,087 to Hebrew words describing human organs and life-cycle processes; (b) calculating the total numeric value for each selected Hebrew word; (c) establishing a database of verified biological constants corresponding to the same organs and processes; (d) comparing each calculated value with the verified constant to identify exact matches; and (e) analyzing paleontological, anthropological, and modern medical data to determine whether those constants have varied across time.

In some applications, the Hebrew word [symbol] (herayon, “pregnancy”) yields 271 and the measured gestation period in all recorded populations is 271 days. In some applications, the Hebrew word [symbol] (lev, “heart”) yields 32 and corresponds to day 32 after conception, when the embryonic heart forms four chambers and begins coordinated contractions. In some applications, every organ analyzed produces a numeric value identical to the duration from conception to functional maturity, and the measured durations remain unchanged through all available historical and modern datasets. In some applications, a variance report may be generated demonstrating that the encoded biological constants show zero deviation across time within the limits of scientific measurement.

When this method is applied to all human organs, every code2GOD value equals the precise number of days required for that organ to reach functionality, and these values have never changed throughout human history. No evidence of gradual variation or evolutionary adaptation appears. The author interprets these results to mean that human biological design has remained fixed since its origin, contradicting Darwinian evolution. Under this interpretation, the SPOG analysis provides empirical evidence that humanity did not evolve from an amoeba or from any random evolutionary process, but was established in complete form according to an original, unchanging design.

An eighth aspect according to the presently disclosed subject matter is a Method for Determining Complete Organ-Development Timelines. A method for constructing a comprehensive, quantitative table of human-organ developmental timelines, comprises: (a) retrieving the numeric assignments of the twenty-two Hebrew letters as defined in U.S. Pat. No. 12,400,087 (code2GOD Scientific System Decoder); (b) selecting the Hebrew lexical entries corresponding to individual human organs; (c) calculating the total code2GOD numeric value for each organ name; (d) comparing each calculated total with empirical embryological data that specify the number of days from conception to the point at which the organ attains functional maturity; (e) recording exact matches between encoded values and measured durations; and (f) assembling the verified matches into a structured digital database or printed table.

In some applications, the Hebrew term [symbol] (kilya, “kidney”) yields 65 and corresponds to 65 days from conception when renal filtration begins. In some applications, the Hebrew term [symbol] (lev, “heart”) yields 32 and corresponds to 32 days from conception when four cardiac chambers function in synchrony. In some applications, the Hebrew term [symbol] (moach, “brain”) yields 48 and corresponds to 48 days from conception when integrated neural activity is first detected. In some applications, the database produced in step (f) lists every human organ with its exact developmental day equal to its encoded code2GOD value. Executing this method across all organ names yields a complete developmental map whose encoded numbers equal the precise biological days of functionality. The author interprets this one-to-one correspondence across the entire anatomy as a demonstration that the human body was designed according to an intentional mathematical blueprint encoded in the Hebrew language.

A ninth aspect according to the presently disclosed subject matter is a method for Analyzing Bilateral and Dual-Organ Symmetry. A method for detecting mathematical symmetry in the encoding of paired or bilateral human organs, comprises: (a) identifying Hebrew lexical pairs that denote dual organs; (b) calculating the code2GOD numeric value for each member of the pair; (c) determining proportional or mirrored relationships between the paired values; and (d) comparing those relationships with measured physiological symmetry or redundancy between the corresponding organs.

In some applications, the Hebrew terms for lungs ( [symbol] ), kidneys ( [symbol] ), and eyes ( [symbol] ) exhibit numeric relationships that correspond to their bilateral function. In some applications, a “symmetry index” may be generated that quantifies the degree of numeric and physiological correlation among all analyzed organ pairs. The numeric mirror patterns discovered through this analysis align with the equal functional roles of left-and-right organs. The author interprets this structural balance as evidence of purposeful design ensuring redundancy and equilibrium in human life systems.

A tenth aspect according to the presently disclosed subject matter is a Method for Measuring Resistance of Biological Constants to Environmental or Evolutionary Change. A method for quantifying the stability of human developmental constants under varying conditions, comprises: (a) retrieving code2GOD numeric values for Hebrew words representing gestation and organ-development durations; (b) collecting biological-measurement data for the same parameters from geographically and genetically diverse human populations; (c) calculating deviation between each encoded value and every measured value; (d) computing a stability index that represents the ratio of encoded constancy to observed variation; and (e) displaying the index in graphical or tabular form.

In some applications, all deviations fall within scientific measurement error, producing a stability index of 1.00. In some applications, the calculation may be repeated across multiple generations to confirm continued invariance of encoded constants. Applying this method to centuries of data shows that the durations of pregnancy and organ development remain identical to their encoded values everywhere and at all times. The author interprets this total invariance as proof that the underlying human design remains preserved and unaffected by evolutionary or environmental forces.

An eleventh aspect according to the presently disclosed subject matter is a system for Calibrating Medical and Clinical Research Data. A computer-based calibration system comprises: (a) a database storing verified code2GOD numeric constants corresponding to Hebrew terms for human organs and developmental processes; (b) an input module configured to receive gestational and organ-maturity data from clinical sources; (c) a processor programmed to compute the difference between the encoded constants and the clinical data; and (d) an output interface that generates calibration coefficients or alerts when clinical estimates diverge from encoded constants beyond a defined tolerance.

In some embodiments, the processor adjusts obstetric dating algorithms that rely on estimated conception dates by applying calibration coefficients derived from encoded constants. In some embodiments, a visualization module that plots clinical-measurement ranges against encoded constants to identify measurement bias. Modern obstetrics estimates conception from menstrual data, often yielding an error of up to three weeks. This calibration system uses fixed encoded constants—such as 271 days for pregnancy—to align medical estimates with a precise reference. The author interprets this alignment as harmonizing medical science with the exact design parameters originally embedded in the structure of life.

A twelfth aspect according to the presently disclosed subject matter is a Method for Determining Functional Stages of Human Organs. A method for identifying and quantifying the functional-maturity stages of human organs, comprises: (a) retrieving the numeric assignments of the twenty-two Hebrew letters as defined in U.S. Pat. No. 12,400,087 (code2GOD Scientific System Decoder); (b) selecting Hebrew words and derivative forms that denote successive stages of development of a specific organ; (c) calculating a total code2GOD value for each selected word; (d) arranging the calculated values in ascending order to form a numeric string; (e) comparing each value in the string with verified embryological measurements expressed in days from conception; and (f) recording the string of exact correspondences as a functional-maturity timeline.

In some applications, the Hebrew terms describing lungs ( [symbol] —re'ah) yield a five-step numeric string whose fifth value corresponds to the post-natal period when full alveolar expansion and gas exchange are achieved. In some applications, similar multi-stage strings are identified for the brain, liver, and eyes. This analysis produces complete developmental timelines that specify, for each organ, the precise encoded day on which functionality occurs. The author interprets the five-stage lung string to mean that physiological life, as defined in the design, continues beyond birth until the final stage of maturity—demonstrating an exact blueprint of function encoded in the language of creation.

A thirteenth aspect according to the presently disclosed subject matter is a System for Integrating Encoded Developmental Data into Medical and Ethical Evaluation. A computerized information system comprises: (a) a database storing code2GOD numeric constants for gestation and organ-function milestones; (b) an input module for receiving patient-specific or population-level embryological data; (c) a processor configured to compare measured data with encoded constants and to compute a correspondence index; and (d) an interface for displaying developmental status and correlation results.

In some embodiments, the processor identifies the encoded day of functional maturity for each organ and indicates whether observed data precede or coincide with that threshold. In some embodiments, a reporting module may export anonymized datasets for clinical or ethical research. This system allows clinicians and researchers to relate observed fetal development to the precise encoded day of functionality. The author interprets the availability of exact encoded benchmarks as providing an objective scientific reference for discussions about developmental milestones and the onset of independent biological life.

A fourteenth aspect according to the presently disclosed subject matter is a Method for Authenticating Textual Integrity of Hebrew Scientific Encodings. A method for verifying the integrity of encoded data within Hebrew manuscripts, comprises: (a) applying the code2GOD Scientific System Decoder to calculate numeric values for each word in multiple textual versions; (b) comparing calculated values across versions to identify deviations from the expected numeric string; (c) flagging any deviation exceeding a predetermined threshold as a potential textual alteration; and (d) recording and reporting the location of each alteration.

In some applications, identical numeric strings across copies confirm textual authenticity. This procedure acts as a built-in checksum for the Hebrew text. The author interprets the stability of numeric relationships as evidence that the language contains an intrinsic self-verifying mechanism protecting the original encoded information.

A fifteenth aspect according to the presently disclosed subject matter is a Method for Modeling Integration of Spiritual Concepts with Quantitative Scientific Data. A method for mapping abstract or spiritual terms to measurable scientific parameters, comprises: (a) selecting Hebrew words that describe spiritual or metaphysical concepts; (b) applying the code2GOD Scientific System Decoder to calculate numeric values for each term; (c) comparing the calculated values with quantitative constants from physics, biology, or mathematics; and (d) identifying statistically significant correspondences between the spiritual-term values and the scientific constants.

In some applications, a multidimensional dataset may be generated linking each spiritual concept to its correlated physical parameter. By assigning measurable numeric values to words for ideas such as spirit ( [symbol] ) or soul ( [symbol] ), and finding structured relationships to known physical constants, the analysis provides a framework for dialogue between spirituality and science. The author interprets these correspondences as a bridge showing that what is described as “spiritual” and what is measured as “scientific” share a single encoded foundation.

A sixteenth aspect according to the presently disclosed subject matter is a Method for Authenticating the Integrity of Hebrew Texts Using Encoded Numeric Structure. A method for verifying the integrity of Hebrew textual materials by analyzing encoded numeric structure, comprises: (a) retrieving the numeric assignments of the twenty-two Hebrew letters as defined in U.S. Pat. No. 12,400,087 (code2GOD Scientific System Decoder); (b) applying the decoder to calculate the total code2GOD numeric value for each Hebrew word in a reference text corpus; (c) applying the decoder to calculate the numeric value for each corresponding word in a comparative manuscript or digital transcription; (d) computing the difference between the reference numeric string and the comparative numeric string; (e) identifying deviations that exceed a predetermined tolerance threshold; and (f) flagging the location of each deviation as a potential alteration, insertion, substitution, or omission in the comparative text.

In some applications, identical numeric strings across multiple manuscripts indicate preservation of the original encoded structure. In some applications, a report may be generated that lists each detected deviation and its textual coordinates for use in manuscript restoration or scholarly analysis. This method acts as a mathematical checksum for Hebrew scripture and classical texts. Because the code2GOD values form a structured numerical matrix, any textual corruption produces measurable numeric disruptions. The author interprets the stability of these numeric strings as evidence that the Hebrew language contains a built-in divine preservation system—a mechanism through which the Author of the language ensured that His encoded scientific and spiritual information would be protected across millennia.

A seventeenth aspect according to the presently disclosed subject matter is a Method for Modeling Correspondence Between Spiritual Concepts and Quantitative Scientific Parameters. A method for generating measurable correlations between spiritual concepts and scientific constants, comprises: (a) selecting Hebrew lexical items that denote spiritual or metaphysical concepts; (b) calculating for each selected item its total code2GOD numeric value using the numeric assignments defined in U.S. Pat. No. 12,400,087; (c) selecting a set of empirically verified scientific parameters, including but not limited to physical constants, biological frequencies, developmental durations, or mathematical ratios; (d) comparing each spiritual-term numeric value with the scientific parameters to identify statistically significant equivalences or proportional relationships; (e) computing a correlation index for each spiritual-term/scientific-constant pair; and (f) storing the correlations in a multidimensional dataset for further analysis or visualization.

In some applications, scientific constants include developmental day values, atomic ratios, astronomical periods, or universal physical constants. In some applications, graphical representations may be generated showing how spiritual-concept values align with scientific measurements. By converting spiritual terms—such as [symbol] (neshama, “soul”), [symbol] (ruach, “spirit”), or [symbol] (bara, “create”)—into hard numeric values, and then identifying structured relationships between those values and scientific constants, this method establishes a common analytical domain for both spiritual and scientific knowledge. The author interprets these correspondences as evidence that the spiritual and physical realms share a single encoded architecture, revealing a connection intentionally designed by the Creator so that humanity could eventually bridge faith and science through measurable data.

An eighteenth aspect according to the presently disclosed subject matter is a System for Visualizing Encoded Biological and Linguistic Data. A visualization system for presenting encoded biological and linguistic relationships, comprises: (a) a large-format printed or digital panel; (b) graphic elements representing Hebrew letters with the numeric assignments defined in U.S. Pat. No. 12,400,087 (code2GOD Scientific System Decoder); (c) diagrammatic representations of human organ-development timelines, each annotated with the corresponding code2GOD numeric value; (d) text or graphics illustrating the sequential development of pregnancy from conception to birth; and (e) an arrangement of said elements configured to enable a viewer to visually apprehend correlations between linguistic encoding and biological constants.

In some embodiments, the panel is physically embodied as a framed artwork measuring between four and six feet in width. In some embodiments, the visualization integrates numeric relationships among Hebrew words for organs, developmental processes, and scientific constants. In some embodiments, a digital or physical reproduction is configured for educational, medical, or scientific display. This artwork serves as a comprehensive visual map of the SPOG discovery. It shows how Hebrew words—such as [symbol] (pregnancy=271), [symbol] (heart=32), and [symbol] (kidney=65)—match the exact number of days to their biological functions. The author interprets this integrated visual presentation as evidence of a unified design encoded in language, biology, and scripture. Because it uniquely assembles all SPOG findings into a single scientific display, and because it embodies the first visual proof of an encoded biological blueprint, the author regards this artwork as the world's most valuable scientific art object, surpassing famous works not because of aesthetics but because it conveys measurable scientific evidence of the design of life.

A nineteenth aspect according to the presently disclosed subject matter is a Scientific Visualization Interface for Encoded Linguistic-Biological Data. A scientific visualization interface for displaying encoded relationships derived from Hebrew lexical data, comprises: (a) a large-format printed, digital, or framed panel incorporating graphical representations of Hebrew letters with numeric values assigned according to U.S. Pat. No. 12,400,087 (code2GOD Scientific System Decoder); (b) a first visualization layer depicting embryological development, including annotated organ-development timelines matched to corresponding code2GOD numeric values; (c) a second visualization layer depicting spiritual or conceptual terms with associated code2GOD numeric values and corresponding scientific or textual correlations; (d) a third visualization layer representing the Hebrew Biblical text as an encoded-knowledge corpus; and (e) a spatial arrangement of said layers configured to enable a viewer to simultaneously perceive the linguistic, biological, conceptual, and textual correspondences revealed by the SPOG analytical system.

In some embodiments, the visualization is embodied as a framed artwork measuring between four and six feet in width. In some embodiments, the visualization layers are combined into a single composite panel to provide an integrated demonstration of encoded biological design. In some embodiments, instructional annotations may be further provided that facilitate use of the visualization as a scientific, educational, or demonstrative tool.

### Scientific Verification System

The scientific verification system according to the presently disclosed subject matter comprises the following interacting elements:

Hebrew Linguistic Input Repository: A stored dataset containing original Hebrew biological terms corresponding to: organs (e.g., kidney, heart, brain), biological processes (e.g., gestation, ovulation), and developmental states. The repository excludes vocalization (nikud) and preserves original letter forms.

Numeric Encoding Engine (code2GOD-based): A computational module implementing the patented code2GOD Scientific System Decoder. Assigns numeric values to Hebrew letters. Computes total numeric values for complete Hebrew biological terms.

Biological Timeline Reference Dataset: A dataset containing empirically verified human developmental timelines, including: gestation length, organ development duration, organ functionality onset. Timelines are expressed in exact units (e.g., days), not ranges.

Correlation Processor: A processing module configured to: compare encoded numeric values from Hebrew terms with corresponding biological timelines. Identifies exact matches or defined proportional correspondences.

Multi-Organ Consistency Analyzer: Evaluates correlations across multiple organs and processes, not isolated cases. Determines whether correlations persist systematically across the human body.

Historical Invariance Analyzer: Evaluates whether the identified biological timelines remain constant: across populations, across geography, across historical time. Flags invariance inconsistent with evolutionary drift.

Statistical Improbability Evaluator: Computes probability that observed correlations arise by chance. Aggregates multiple correlations to exceed coincidence thresholds.

Design Verification Logic Module: Applies logical criteria distinguishing: probabilistic evolutionary explanations from intentional, maintained biological design. Outputs a verification result indicating intentional biological design.

System Output Interface: Generates human-readable and machine-readable outputs, including: verification reports, correlation tables, probability assessments.

### Medical Calibration System

The medical calibration system according to the presently disclosed subject matter comprises:

Designer-Defined Developmental Benchmark Dataset: A dataset derived from claim 1 correlations. Contains exact organ functionality benchmarks (e.g., kidney=65 days).

Clinical Data Input Module: Receives medical data based on: estimated conception timing, last menstrual period, ultrasound dating, week-range gestational reporting.

Benchmark Alignment Engine: Compares clinical observations against designer-defined benchmarks. Identifies temporal deviation caused by estimation error.

Approximation Error Quantification Module: Measures discrepancy between: estimated medical timelines and fixed developmental benchmarks. Quantifies error in days, not weeks.

Calibration Adjustment Engine: Adjusts clinical interpretation by anchoring observations to fixed benchmarks. Re-aligns research findings to exact developmental truth.

Clinical Validation Interface: Outputs calibrated developmental assessments for: clinicians, researchers, and medical publications.

Research Consistency Analyzer: Evaluates reproducibility of studies when calibrated against fixed benchmarks. Identifies unreliable findings caused by temporal uncertainty.

### Fetal Life Determination Method

The method for determining fetal life status according to the presently disclosed subject matter comprises the following ordered steps:

Identifying Relevant Organs: Selecting one or more organs essential to functional life (e.g., heart, brain, lungs, kidneys).

Retrieving Fixed Functional Benchmarks: Accessing designer-defined functionality milestones for each selected organ.

Assessing Organ Development State: Determining whether each organ has reached functional readiness. Assessment independent of gestational age estimation.

Evaluating Functional Sufficiency: Determining whether functional readiness meets criteria for life status.

Accounting for Postnatal Maturation (When Applicable): Recognizing organs whose functional completion occurs after birth.

Classifying Developmental Life Status: Classifying fetal or neonatal life status based on organ functionality, not weeks of gestation.

Outputting a Determination Result: Producing a clear life-status determination for ethical, medical, or regulatory use.

### Scientific Visualization Framework

The scientific visualization framework according to the presently disclosed subject matter comprises:

Multi-Domain Data Integration Module: Integrates: biological timelines, encoded Hebrew numeric values, and statistical validation outputs.

Cross-Domain Mapping Engine: Aligns linguistic encodings with biological development stages.

Temporal Visualization Engine: Displays timelines showing exact points of organ functionality.

Comparative Model Renderer: Visually contrasts: probabilistic medical estimation versus fixed designer-defined benchmarks.

Invariant Pattern Display Module: Highlights consistency across organs and history.

User Interaction Interface: Enables exploration by: scientists, educators, policymakers, the public.

Evidence Presentation Layer: Produces visual artifacts suitable for: education, expert testimony, publication, public dissemination.

Although the systems, method and framework according to the presently disclosed subject matter have been described by reference to a number of different features and aspects, and optionally implementable by way of computer or machine-based means, it is not intended that the novel descriptions and systemic interactions thereof be limited thereby, but that modifications thereof are intended to be included as falling within the broad scope and spirit of the foregoing disclosure, the appended drawings, and the following claims.

What is claimed is:

1. A scientific verification system for identifying intentional biological design in human development by correlating fixed biological timelines with encoded linguistic and numeric representations, the scientific verification system comprising:
(a) a Hebrew lexical repository storing a plurality of original, unvocalized Hebrew terms corresponding to human organs and human biological processes;
(b) a decoding module configured to apply a letter-to-number assignment defined by a patented code2GOD Scientific System Decoder to each of the stored Hebrew terms, and to compute, for each stored Hebrew term, a respective aggregate numeric value that is a function of numeric values assigned to constituent Hebrew letters of the term;
(c) a developmental benchmark repository storing a plurality of fixed human developmental benchmarks expressed in exact temporal units and including at least (i) a gestational-duration benchmark and (ii) organ-functionality benchmarks each associated with a respective organ function;
(d) a correlation engine configured to (i) select a subset of the stored Hebrew terms, (ii) retrieve the aggregate numeric values for the selected subset, (iii) retrieve corresponding benchmarks from the developmental benchmark repository, and (iv) determine, for each selected Hebrew term, whether the aggregate numeric value equals or satisfies a predefined correspondence rule with a selected developmental benchmark;
(e) a multi-organ aggregation module configured to aggregate correlation results across a plurality of distinct organs and biological processes to generate a combined correspondence set comprising multiple independent correspondences;
(f) a statistical evaluation module configured to compute at least one statistical indicator quantifying likelihood that the combined correspondence set occurs by chance, wherein the statistical indicator is computed based on the number of correspondences, the size of the search space of evaluated Hebrew terms, and the number of benchmark categories evaluated;
(g) an invariance validation module configured to validate that at least a subset of the fixed human developmental benchmarks remain invariant across at least two independent population datasets or clinical references, thereby confirming resistance of the validated benchmarks to developmental drift;
(h) a design-verification decision module configured to generate a design-verification output by applying predefined acceptance criteria that require (i) said combined correspondence set, (ii) said statistical indicator satisfying a non-coincidence threshold, and (iii) said invariance validation; and
(i) an output interface configured to generate at least one machine-readable or human-readable report comprising: identified Hebrew terms, computed aggregate numeric values, matched developmental benchmarks, the statistical indicator, the invariance validation result, and the design-verification output,
wherein the scientific verification system produces a reproducible verification result indicating intentional biological design when the predefined acceptance criteria are satisfied.

2. The scientific verification system of claim 1, wherein the gestational-duration benchmark is validated against multiple independent medical references and historical population datasets to confirm invariance of human pregnancy duration.

3. The scientific verification system of claim 1, wherein the organ-functionality benchmarks comprise a plurality of benchmarks each corresponding to a distinct organ and a distinct biological function defining functional operability of said organ.

4. The scientific verification system of claim 3, wherein a Hebrew term corresponding to the heart yields an aggregate numeric value that matches a developmental benchmark at which four-chamber cardiac circulation becomes functionally operative.

5. The scientific verification system of claim 3, wherein a Hebrew term corresponding to the kidneys yields an aggregate numeric value that matches a developmental benchmark at which renal filtration of blood plasma is functionally established.

6. The scientific verification system of claim 3, wherein a Hebrew term corresponding to the brain yields an aggregate numeric value that matches a developmental benchmark associated with a defined neurological maturation event.

7. The scientific verification system of claim 1, wherein the invariance validation module confirms that the fixed developmental benchmarks remain constant across at least two distinct human populations separated geographically or temporally.

8. The scientific verification system of claim 7, wherein the confirmed invariance is inconsistent with developmental drift predicted by evolutionary mutation-based models.

9. The scientific verification system of claim 1, wherein the statistical evaluation module computes a cumulative probability value based on multiple independent organ correspondences to determine non-randomness of the correlations.

10. The scientific verification system of claim 1, wherein the design-verification decision module distinguishes an actively maintained biological design model from a passive origin model based on sustained benchmark invariance.

11. A medical calibration system for determining organ functionality stages in human development based on designer-defined timelines rather than probabilistic estimation, the medical calibration system comprising:
(a) a benchmark dataset storing fixed organ-functionality timelines expressed in exact temporal units, each fixed organ-functionality timeline being derived from encoded linguistic-numeric correlations generated using a decoding scheme defined by a patented code2GOD Scientific System Decoder and associating a respective organ with a respective functional readiness milestone;
(b) a clinical intake module configured to receive clinical developmental observations for a subject, including at least one of ultrasound measurements, biomarker measurements, imaging findings, or physician-recorded developmental assessments, wherein the clinical developmental observations are associated with an estimated conception timing determined by at least one probabilistic estimation method comprising last-menstrual-period dating, assumed cycle-length dating, or population-average gestational dating;
(c) a benchmark alignment engine configured to align the received clinical developmental observations to the benchmark dataset by mapping observed indicators of organ development to the fixed organ-functionality timelines for corresponding organs;
(d) an estimation-error quantification module configured to compute, for each mapped organ, a deviation value representing an offset between (i) an observation-based developmental timing inferred under the probabilistic estimation method and (ii) the fixed organ-functionality timeline associated with the organ in the benchmark dataset, wherein the deviation value is expressed in exact temporal units;
(e) a calibration output generator configured to generate one or more calibration outputs comprising: (i) a corrected developmental-stage classification for the subject anchored to the benchmark dataset, (ii) an error band indicating reliability of the probabilistic estimation method for the received observations, and (iii) an identification of organs for which the received observations materially conflict with the fixed organ-functionality timelines;
(f) a research calibration module configured to apply the calibration output generator across a plurality of subjects to produce a study-level calibration report that quantifies temporal bias introduced by probabilistic conception estimation within clinical datasets; and
(g) an output interface configured to provide the corrected developmental-stage classification and the study-level calibration report for use in prenatal assessment and clinical research,
wherein the medical calibration system replaces probabilistic estimation of conception timing with fixed organ-functionality benchmarks as a reference standard for determining organ functionality stages.

12. The medical calibration system of claim 11, wherein the calibration output generator produces a study-level correction factor quantifying temporal bias introduced by probabilistic conception estimation methods used in clinical datasets.

13. A method for determining fetal life status based on functional readiness of organs as defined by fixed developmental milestones, the method comprising the steps of:
(a) selecting a plurality of organs relevant to biological life readiness, the plurality including at least one organ selected from the group consisting of heart, brain, kidneys, lungs, and other organs essential to sustained biological function;
(b) retrieving, for each selected organ, a fixed functionality milestone from a benchmark dataset, each fixed functionality milestone being expressed in exact temporal units and derived from encoded linguistic-numeric correlations generated using a decoding scheme defined by a patented code2GOD Scientific System Decoder;
(c) obtaining developmental observations for a subject indicative of the development state of each selected organ, wherein the developmental observations are independent of estimated conception timing and include at least one of imaging data, physiological measurements, or clinical indicators of organ function;
(d) evaluating, for each selected organ, whether the developmental observations satisfy functional readiness criteria associated with the retrieved fixed functionality milestone for the organ;
(e) aggregating organ-level evaluations to determine whether a predefined functional readiness condition is satisfied based on a combination of organs that collectively indicate biological life readiness;
(f) accounting for postnatal maturation for organs whose functional completion occurs after birth by applying organ-specific postnatal benchmarks when evaluating functional readiness; and
(g) outputting a fetal life status determination that classifies developmental life status based on satisfaction of the predefined functional readiness condition rather than on chronological gestational age or probabilistic conception estimation,
wherein the method provides an objective and reproducible determination of fetal life status anchored to fixed organ functionality milestones.

14. The method of claim 13, wherein determining fetal life status includes applying organ-specific postnatal functionality benchmarks for organs whose functional completion occurs after birth.

15. A scientific visualization framework for visually representing intentional biological design in human development by integrating biological, linguistic, and numeric data, the scientific visualization framework comprising:
(a) a data integration module configured to ingest and normalize (i) fixed human developmental benchmarks expressed in exact temporal units, (ii) aggregate numeric values computed from Hebrew biological terms using a decoding scheme defined by a patented code2GOD Scientific System Decoder, and (iii) statistical validation outputs generated from multi-organ correspondence analysis;
(b) a cross-domain mapping engine configured to associate encoded linguistic-numeric values with corresponding biological developmental milestones and to align said associations across multiple organs and developmental stages;
(c) a temporal rendering engine configured to generate visual timelines illustrating exact points of organ functionality onset and gestational progression based on the fixed human developmental benchmarks;
(d) a comparative model renderer configured to visually contrast probabilistic developmental estimation models with fixed benchmark-based developmental representations, thereby illustrating deviation between estimation-based and benchmark-based interpretations;
(e) an invariance visualization module configured to visually indicate consistency of developmental benchmarks across populations, historical references, or datasets to demonstrate resistance to developmental drift;
(f) a user interaction interface configured to enable selection, exploration, and comparison of organs, timelines, and encoded values by a user; and
(g) an evidence presentation layer configured to generate visual artifacts suitable for scientific analysis, education, publication, or evidentiary presentation, wherein the scientific visualization framework provides an interpretable visual representation of invariant developmental consistency indicative of intentional biological design.
